(19) United States
(12) Patent Application Publication
(10) Pub. No.: US 2025/0022537 A1
(43) Pub. Date: Jan. 16, 2025

(54) MOLECULAR SUBTYPING METHOD FOR GASTRIC CANCER BASED ON METABOLIC GENES, CONSTRUCTION METHOD FOR PROGNOSTIC PREDICTION MODEL AND APPLICATIONS

(71) Applicants: Shandong First Medical University Affiliated Provincial Hospital (Shandong Provincial Hospital), Jinan (CN); Shandong University Qilu Hospital, Jinan (CN)

(72) Inventors: Leping Li, Jinan (CN); Wei Chong, Jinan (CN); Hao Chen, Jinan (CN); Liang Shang, Jinan (CN); Xingyu Zhu, Jinan (CN); Kang Xu, Jinan (CN)

(21) Appl. No.: 18/772,065

(22) Filed: Jul. 12, 2024

(30) Foreign Application Priority Data
Jul. 13, 2023 (CN) 202310859047.2

(51) Int. Cl. G16B 25/10; G16B 40/30; G16H 50/70

(52) U.S. Cl. CPC G16B 25/10; G16B 40/30; G16H 50/70

(57) ABSTRACT

The present disclosure belongs to the technical fields of computational medicine and clinical oncology, and specifically relates to a molecular subtyping method for gastric cancer (GC) based on metabolic genes, a construction method for a prognostic prediction model and applications. In the present disclosure, metabolic process alterations are overall assessed, and new metabolic transcription patterns based on 456 metabolic gene-related signatures are identified. Three different metabolic patterns are validated by unsupervised consensus clustering analysis and independent datasets, and molecular and clinical pathological features of these three metabolite clusters have different metabolic gene expressions, pathway enrichments, gene variations, and survival outcomes. In addition, in the present disclosure, a scoring scheme is constructed to assess metabolic patterns of individual tumors, revealing the relationships between the metabolic patterns with prognosis, cuproptosis and immune regulation, and having a good value for practical application.

### CROSS-REFERENCE TO RELATED APPLICATIONS

This application claims priority of Chinese Patent Application No. 202310859047. 2, filed on Jul. 13, 2023, the entire contents of which are incorporated herein by reference.

### TECHNICAL FIELD

The present disclosure belongs to the technical fields of computational medicine and clinical oncology, and specifically relates to a molecular subtyping method for gastric cancer (GC) based on metabolic genes, a construction method for a prognostic prediction model and applications.

### BACKGROUND

GC remains the fifth most common malignant tumor worldwide and has a poor prognosis due to high recurrence rates in early stages and limited treatment strategies. The critical role of metabolic reprogramming in the development and metastasis of GC has recently been recognized and presents opportunities for diagnosis, treatment and prognosis of cancer. It is difficult to understand metabolic landscape due to the diversity and heterogeneity of metabolic alterations in GC. Some previous studies have applied only GC cell strains to investigate the metabolic microenvironment that regulates abnormalities in GC, and other previous studies have primarily focused on the pan-cancer analysis of metabolic dysregulation based on genomic perturbation, leading to a poor understanding of the metabolic heterogeneity of GC.

The metabolic features of onset and evolution of GC remain obscure due to the heterogeneous metabolic preferences and dependencies of tumors. In addition, it has been confirmed that immunotherapies targeting specific immune checkpoint proteins, such as pembrolizumab or nivolumab, which target PD-1, have significant clinical utility in GC. However, the relationship between metabolism alteration of tumor and immune response of GC remains unclear.

### SUMMARY

In response to the above deficiencies, the present disclosure provides a molecular subtyping method for GC based on metabolic genes, a construction method for a prognostic prediction model and applications. The present disclosure is realized by the following technical solutions.

In a first aspect of the present disclosure, a molecular subtyping method for GC based on metabolic genes is provided, including the following steps:
S1, acquiring gene/protein expression profiling data and clinical information data of patients with GC;
S2, acquiring GC metabolism-related genes and GC metabolite-related features formed thereby on the basis of established studies; and
S3, performing consensus unsupervised clustering using a non-negative matrix factorization (NMF) algorithm, specifically, identifying different metabolic patterns on the basis of the GC metabolite-related features formed by the GC metabolism-related genes; decomposing a metabolic gene profile into two non-negative matrices, W and H; repeating factorization of a matrix A, and aggregating outputs thereof, to obtain consensus clusters of GC samples, classifying GC into three heterogeneous clusters with different metabolic features, prognoses, proteogenomic variants and metabolites, i.e. three molecular subtypes, named metabolic signature cluster (MSC) 1, MSC2 and MSC3.

In a specific embodiment of the present disclosure, MSC1 has a better prognosis, up-regulates lipogenesis, glutathione metabolism, and oxidative metabolism, and has a moderate immune infiltration status and mismatch repair (MMR); MSC2 has a moderate prognosis, enriched amino acid and nucleotide metabolism, and has a highly defective MMR status, and immune-enriched subtypes; and MSC3 has a poorer prognosis, enhances saccharide and carbohydrate metabolism, and has highly mesenchymal and infiltrative features.

In step S1, sources of the gene/protein expression profiling data and clinical information data of patients with GC include, but are not limited to, national center of biotechnology information-gene expression omnibus (NCBI-GEO), the cancer genome atlas (TCGA), clinical proteomic tumor analysis consortium (CPTAC), and actual clinical samples.

The specific method for step S2 includes:
collating a gene set of metabolic super-pathways, including metabolic genes for amino acid, carbohydrate, energy, glycan, lipid, nucleotide, tricarboxylic acid (TCA), and vitamin cofactor, according to the annotation of a biomolecular pathways knowledge base (Reactome); utilizing networks of collected metabolite-protein interactions (MPIs) and considering the linkage between metabolite-interacting genes and four or more networks of MPIs as an effective metabolite subgroup; and performing differential expression analysis between TCGA tumors and adjacent normal tissues for overlapped genes in a dataset, and finally identifying GC metabolite-related features formed by a plurality of metabolic genes.

In step S3, the three heterogeneous clusters are determined according to a correlation coefficient, a residual sum of squares (RSS) coefficient, a dispersion coefficient and a silhouette coefficient.

In a second aspect of the present disclosure, a construction method for a GC prognostic model based on the subtyping method in the first aspect is provided, including the following steps:
S1, acquiring overlapped differentially expressed genes (DEGs) among the three MSC subtypes on the basis of the above subtyping method, and performing prognostic analysis of each gene using a univariate Cox regression model;
S2, extracting genes with significant prognostic significance using a random forest algorithm; and
S3, performing principal component analysis (PCA), performing matrix multiplication of the standardized dataset and feature vectors to obtain PC scores, and taking a sum of scores of first three PCs, PC1, PC2, and PC3, as a metabolic subtype-related prognostic gene score (MSPG-score): MSPG=Σ(PC1+PC2+PC3).

In step S2, the genes include tetratricopeptide repeat domain 28 (TTC28), glycoprotein A33 (GPA33), phosphodiesterase 7B (PDE7B), sodium voltage-gated channel beta subunit 4 (SCN4B), lamin (LMNB2), hepatocyte nuclear factor 4 gamma (HNF4G), galectin (LGALS3), secreted protein acidic and rich in cysteine-like 1 (SPARCL1), cytidine diphosphate-diacylglycerol synthase 1 (CDS1), zinc finger protein 532 (ZNF532), and furry (FRY).

In a third aspect of the present disclosure, one or more of applications of the molecular subtyping method for GC, the construction method, or a GC prognostic model obtained by the construction method are provided as follows:
(a) studies on biological mechanisms related to GC;
(b) prognostic assessment of patients with GC or preparation of products for the prognostic assessment of patients with GC;
(c) related genomic variation studies;
(d) proteomic and phosphoproteomic studies;
(e) prediction of immune response and therapeutic benefit from the therapy using an immune checkpoint inhibitor (ICI) or preparation of products predicting immune response and therapeutic benefit from the therapy using ICI;
(f) studies on copper-related proteins of GC cells; and
(g) studies on drug sensitivity of GC cells.

In the (b), the prognostic assessment of patients with GC includes at least survival assessment of the patients with GC.

In the (e), the ICI is specifically a PD-1/PD-L1 inhibitor.

In the (f), the copper-related proteins of GC cells include, but are not limited to, ferredoxin 1 (FDX1), lipoic acid synthetase (LIAS), dihydrolipoamide dehydrogenase (DLD), dihydrolipoamide S-succinyltransferase (DLST), dihydrolipoamide S-acetyltransferase (DLAT), pyruvate dehydrogenase E1 subunit alpha 1 (PDHA1), pyruvate dehydrogenase E1 subunit beta (PDHB), and glutaminase (GLS) proteins.

In the (g), the drug includes Foretinib, PIK-93, AT7867, Imatinib, Dasatinib and Y-27632.

In the (f) and (g), the GC cells include KATO-3 and HGC27.

Beneficial technical effects of one or more of the above technical solutions are as follows.

In the above technical solutions, metabolic transcriptomic makers are utilized to identify three different metabolic patterns in GC. By means of comprehensive multi-omics analysis, specific metabolic pathways, clinicopathological features, immune infiltration and genomic mutations associated with these three metabolic patterns are characterized. In addition, in the above technical solutions, a quantitative system is further established, named “MSPG-score”, to more accurately assess metabolic features of individual patients. Metabolic marker subtypes and metabolite dysregulation are validated using a GC group containing paired transcriptomic and metabolomic data. A comprehensive understanding of the molecular and biological features of tumor metabolism will expand the understanding of metabolic reprogramming in tumorigenesis and provide potential markers for prognostic assessment and sensitivity treatment of GC, and thus has a good value for practical application.

### BRIEF DESCRIPTION OF THE DRAWINGS

FIG. 1A shows a flowchart related to construction plans for GC metabolite patterns and features.

FIG. 1B shows that a total of 456 DEGs representing key differentiating indicators of GC metabolic features are considered as metabolic feature indicators, as shown in a VENN diagram.

FIG. 2A shows the metabolic signature-based clustering results for GC samples in Asian cancer research group (ACRG) cohort, where the normalized gene expression profiles of metabolic feature genes in eight metabolic super-pathways in three MSC subtypes are showed, and clinical variables and molecular features corresponding to each sample are plotted at the top of the map.

FIG. 2B shows that the overlay of metabolic feature-based subtypes (inner ring) with the known GC molecular subtypes or clinical features in TCGA (top) and Singapore cohorts (bottom). The TCGA cohort includes integrative molecular subtype (outer ring), immune subtype (middle ring), and tumor microenvironment (TME) subtype (inner ring), and the distribution of these subtypes and MSC are shown via a bar graph; and the Singapore cohort includes molecular subtype (outer ring), survival status (middle ring), and Lauren classification (inner ring), and the distribution of these subtypes and MSC are shown via a bar graph; and statistical differences between the three clusters are compared using chi-square test or corrected parity check. *P<0.05, **P<0.01, and ***P<0. 001.

FIG. 2C shows Kaplan-Meier curves for overall survival of the three MSC subtypes of GC in ACRG, TCGA-stomach adenocarcinoma (STAD), and Singapore cohort (Log-rank test, P<0.05).

FIG. 3A shows gene set variation analysis (GSVA) of three MSC subtypes in ACRG, TCGA and Singapore datasets, and the heat map shows the normalized mean enrichment scores of Kyoto encyclopedia of genes and genomes (KEGG) pathways enriched in the three MSC subtypes.

FIG. 3B shows metabolic genes of glycolysis, TCA cycle, fatty acid biosynthesis (FAs), and arachidonic acid (AA). Mean messenger ribonucleic acid (mRNA) expression is significantly up- or down-regulated in ACRG cohort relative to the MSC1 and MSC3 subtypes (the gene expression level of MSC2 is normalized to zero); and the color module below each gene is depicted as log ratios (fold change (FC), and a ratio of the mean mRNA expression of MSC1 or MSC3 subtype to MSC2 subtype is expressed using log 2).

FIG. 3C shows the comparison of cuproptosis, FDX1, tumor ploidy, and MMR levels among different MSC subtypes.

FIG. 4A shows a flowchart of the construction of an MSPG scoring system for GC, a total of 592 DEGs in the three MSC subtypes are subjected to one-way Cox regression analysis, and further subjected to feature selection analysis and PCA analysis using prognosis-related genes.

FIG. 4B shows the distribution of MSPG-score for different MSC subtypes in ACRG, TCGA and Singapore cohorts, and Kruskal-Wallis test is used to compare the differences among these three subgroups.

FIG. 4C is a heat map showing the highest enriched KEGG biopathway of MSPG KEGG calculated by the GSVA algorithm.

FIG. 4D shows the correlation between MSPG-score and known biosignatures studied by Zeng et al.

FIG. 4E shows the identification of cutpoints for MSPG-score subgroups in GC, and the MSPG-score with the highest standardized log-rank statistic (1. 4041) is taken as the optimal cutpoint.

FIG. 4F shows survival analyses for the low MSPG-score and high MSPG-score subgroups of patients with GC across independent datasets.

FIG. 4G is an alluvial plot of MSC subtypes in different molecular subtypes, MSPG-score subgroups, and survival status groups.

FIG. 5A shows the correlation between MSPG-score and clinical and molecular features in an anti-pd-1 treatment cohort in GC.

FIG. 5B shows the patients with MSC1 subtype have significant therapeutic effect and immune response compared to other patients treated with anti-pd-1.

FIG. 5C shows the patients with high MSPG-score have significant therapeutic effect and immune response compared to other patients treated with anti-pd-1.

FIG. 5D shows distribution patterns of MSPG-score in different PD-L1 status, showing higher MSPG-score in PD-L1-positive group.

FIG. 5E shows distribution patterns of MSPG-score in different Epstein-Barr virus (EBV) status, showing higher MSPG-score in EBV positive group.

FIG. 5F shows the efficacy of MSPG-score, microsatellite instability (MSI)/microsatellite stability (MSS), and the combined predictive indicators of the two indicators in predicting immune response by ROC curves, and statistical tests are performed using the two-sided DeLong test.

FIG. 5G shows Kaplan-Meier curves for high MSPG-score and low MSPG-score subgroups in melanoma anti-pd-1 immunotherapy cohort (left), and percentage of clinical responses to anti-pd-1 therapy in different MSPG-score subgroups (right).

FIG. 5H shows Kaplan-Meier curves for patients with high and low MSPG-score in urothelium carcinoma (UC) anti-PD-L1 immunotherapy cohort (left), and percentage of clinical responses to anti-PD-L1 immunotherapy in different MSPG-score groups (right).

FIG. 6A shows transcriptomic and metabolomic metabolic feature profiles of the MSPG and MSC subgroups in internal provincial hospital GC cohort (n=33).

FIG. 6B shows the comparison of MSPG-score, FDX1 expression, cuproptosis, and MMR scores among different MSC subtypes.

FIG. 6C shows the correlation between MSPG-score and the known biosignatures studies by Zeng et al. through Spearman analysis.

FIG. 6D is a network module showing the linkage between MSPG-score subgroup-related metabolic genes and enriched KEGG biomodules. The size of nodes in the center of the module represents the number of enriched genes.

FIG. 6E shows pathway-based analysis of metabolomic changes between high and low MSPG-score subgroups. The differential abundance (DA) score captures the average, and total change in all metabolites in a pathway. A score of 1 indicates that all measured metabolites in a pathway increase in the high subgroup compared to the low subgroup, and a score of-1 indicates that all measured metabolites decrease in a pathway

FIG. 7A shows unsupervised clustering of metabolite gene signatures in the ACRG dataset using NMF algorithm in an example of the present disclosure. The heat map (top) represents NMF clusters of 456 metabolite gene expression profiles, with clusters numbered from 2 to 6. The line graph (bottom) represents the relationship between cophenetic, consensus trends, evar, residuals, and silhouette coefficients associated with the number of clusters.

FIG. 7B shows unsupervised clustering of metabolite gene signatures in the TCGA dataset using NMF algorithm in an example of the present disclosure. The heat map (top) represents NMF clusters of 456 metabolite gene expression profiles, with clusters numbered from 2 to 6. The line graph (bottom) represents the relationship between cophenetic, consensus trends, evar, residuals, and silhouette coefficients associated with the number of clusters.

FIG. 7C shows unsupervised clustering of metabolite gene signatures in the Singapore dataset using NMF algorithm in an example of the present disclosure. The heat map (top) represents NMF clusters of 456 metabolite gene expression profiles, with clusters numbered from 2 to 6. The line graph (bottom) represents the relationship between cophenetic, consensus trends, evar, residuals, and silhouette coefficients associated with the number of clusters.

FIG. 8A is a heat map showing normalized gene expression profiles of metabolic marker genes of eight metabolic super-pathways among the three MSC subtypes in the TCGA dataset. The clinical variables and molecular features corresponding to each sample are plotted at the top of the map.

FIG. 8B is a heat map showing normalized gene expression profiles of metabolic marker genes of eight metabolic super-pathways among the three MSC subtypes in the Singapore dataset. The clinical variables and molecular features corresponding to each sample are plotted at the top of the map.

FIG. 8C shows a schematic diagram of the identification of metabolomics and transcriptomics-estimated subtypes in GC cell lines.

FIG. 8D shows that three clusters are identified by metabolite-based consensus clustering in GC cell lines.

FIG. 8E shows the comparison of the abundance of specific metabolites among the three metabolomics subtypes.

FIG. 8F is bar graphs showing the distribution of the molecular subtypes, EBV infection, and MSI status among metabolite-marker-based subtypes for each sample in ACRG cohort.

FIG. 8G shows the clinical prognostic value of different MSC subgroups estimated by subgroup analysis via multifactor Cox regression in ACRG, TCGA, and Singapore cohorts. The length of the horizontal line indicates the 95% confidence interval for each group.

FIG. 9A shows the feature selection process for 204 prognosis-related genes based on the random forest algorithm.

FIG. 9B presents RNA interference (RNAi) scores and CERES scores for 11 genes.

FIG. 9C shows a scree plot of extracted eigenvalues by PCA analysis, in a descending order.

FIG. 9D shows relative expression levels of 11 MSPG-score-related genes in the three MSC subtypes.

FIG. 9E shows the direction of variance of PCs of the 11 extracted MSPG-score-related genes.

FIG. 9F is AUC showing the advantage of MSPG-score in distinguishing the three metabolic marker subtypes.

FIG. 9G shows the correlation between MSPG-score and expression of cuproptosis-related molecules in GC.

FIG. 9H is ROC showing the survival prediction advantage of the established MSPG-score model in ACRG cohort.

FIG. 9I shows the clinical prognostic value of different MSPG-score subgroups in ACRG, TCGA, and Singapore cohorts estimated by subgroup analysis using multifactor Cox regression. The length of the horizontal line indicates the 95% confidence interval for each group.

### DETAILED DESCRIPTION

### Example 1

### 1. Method

### Collection and Pre-Processing of Publicly Available Datasets

Multi-omics sequencing data and clinical annotations for GC samples were collected retrospectively from the NCBI-GEO (https://www.ncbi.nlm.nih.gov/geo/), TCGA (https://cancergenome.nih.gov/) and CPTAC database (https://cptac-data-portal.georgetown.edu/cptac) databases.

Ultimately, a total of 900 patients with GC were included in this analysis, including those from a GSE62254/ACRG cohort (N=300), a GSE15459/Singapore cohort (N=192), TCGA-STAD (N=375), and a Shandong Provincial hospital (SDPH)-GC cohort of (N=33). TCGA RNA sequencing data (FPKM format) was downloaded from the UCSC Xena database (https://gdc.xenahubs.net/download/TCGA-STAD.htseq_fpkm.tsv.gz). In addition, gene-level proteomic and phosphoproteomic data were downloaded from the CPTAC website (https://proteomics.cancer.gov/programs/cptac), and protein abundance was subjected to log 2 logarithmic transformation and normalized by median.

Collection and preparation of SDPH clinical specimens

| | | | | Tumor | Tumor node | | | | | |
|---|---|---|---|---|---|---|---|---|---|---|
| | | | | differentiation | metastasis | | | | | |
| GC | | | | degree (high, | (TNM) | Clinical | MSI/ | Pathological | MSC | |
| sample | Gender | Age | Tumor site | middle, and low) | classification | stage | MSS | type | subtype | MSPG-score |
| T01 | Female | 64 | Posterior wall | Low | T4bN1M0 | IIIB | MSI | Adenocarcinoma | MSC2 | −0.982251191 |
| | | | of gastric antrum | differentiation | | | | | | |
| T02 | Male | 57 | Gastric antrum | Low | T4aN0M0 | IIB | MSS | Adenocarcinoma, | MSC1 | 0.380849695 |
| | | | | differentiation | | | | and most are | | |
| | | | | | | | | signet-ring | | |
| | | | | | | | | cell carcinoma | | |
| T03 | Male | 65 | Gastric antrum | Middle-low | T4bN3M1 | IV | MSS | Adenocarcinoma, | MSC2 | 1.63851506 |
| | | | | differentiation | | | | and | | |
| | | | | | | | | neuroendocrine | | |
| | | | | | | | | neoplasm | | |
| T04 | Male | 60 | Gastric angle | Middle-low | T1N1M0 | IB | MSS | Mucinous | MSC3 | −4.263700882 |
| | | | | differentiation | | | | adenocarcinoma | | |
| T05 | Male | 65 | Cardia | Low | T4aN3aM0 | IIIC | MSS | Adenocarcinoma | MSC2 | 0.241705556 |
| | | | | differentiation | | | | | | |
| T06 | Male | 50 | Gastric antrum | Low | T4bN0M0 | IIIA | MSS | Adenocarcinoma | MSC2 | −0.180438783 |
| | | | | differentiation | | | | | | |
| T07 | Female | 57 | Gastric antrum | Low | T4bN3aM1 | IV | MSS | Adenocarcinoma | MSC3 | −4.004454328 |
| | | | | differentiation | | | | | | |
| T08 | Male | 69 | Gastric antrum | Signet-ring | T4bN0M0 | IIIB | MSS | Signet-ring | MSC2 | −1.909533273 |
| | | | | cell carcinoma | | | | cell carcinoma | | |
| T09 | Male | 55 | Gastric antrum | Low | T4bN3aM1 | IV | MSS | Adenocarcinoma | MSC1 | 2.927034742 |
| | | | | differentiation | | | | | | |
| T10 | Female | 70 | Gastric antrum | Low | T4bN2M0 | IIIB | MSS | Adenocarcinoma | MSC2 | 1.971132287 |
| | | | | differentiation | | | | | | |
| T11 | Male | 57 | Gastric antrum | Middle | T4aN2M0 | IIIB | MSS | Adenocarcinoma | MSC3 | −5.971268913 |
| | | | | differentiation | | | | | | |
| T12 | Female | 76 | Lesser curvature | Low | T4aN1M0 | IIIA | MSS | Adenocarcinoma | MSC2 | 3.468593329 |
| | | | of stomach | differentiation | | | | | | |
| T13 | Male | 80 | Gastric antrum | Middle | T4bN1M0 | IIIC | MSS | Adenocarcinoma | MSC1 | 4.811507166 |
| | | | | differentiation | | | | | | |
| T14 | Male | 51 | Cardia | Low | T3N1M0 | IIB | MSS | Adenocarcinoma | MSC2 | −0.550513126 |
| | | | | differentiation | | | | | | |
| T15 | Male | 70 | Gastric antrum | Low | T4bN3cM1 | IV | MSS | Adenocarcinoma | MSC2 | 1.355069528 |
| | | | | differentiation | | | | | | |
| T16 | Female | 74 | Pre-pyloric area | Low | T2N3M0 | IIIA | MSS | Adenocarcinoma | MSC2 | −1.235752314 |
| | | | | differentiation | | | | | | |
| T17 | Male | 57 | Gastric antrum | Non | T4aN3bM0 | IIIC | MSS | Adenocarcinoma | MSC2 | 2.092039478 |
| T18 | Male | 29 | Gastric antrum | Low | T4aN3M0 | IIIC | MSS | Adenocarcinoma | MSC3 | −5.372673226 |
| | | | | differentiation | | | | | | |
| T19 | Female | 42 | Lesser curvature | Low | T4aN3M0 | IIIC | MSS | Adenocarcinoma | MSC1 | 1.720811129 |
| | | | of stomach body | differentiation | | | | | | |
| T20 | Male | 59 | Gastric angle | Low | T4aN1M0 | IIIA | MSS | Adenocarcinoma | MSC2 | 1.951643924 |
| | | | | differentiation | | | | | | |
| T21 | Male | 58 | Gastric antrum | Low | T4aN3aM0 | IIIC | MSS | Adenocarcinoma | MSC1 | 3.683535436 |
| | | | | differentiation | | | | | | |
| T22 | Female | 71 | Linitis plastica, | Low | T4aN3aM0 | IIIC | MSS | Adenocarcinoma | MSC1 | 4.399836512 |
| | | | and whole stomach | differentiation | | | | | | |
| T23 | Male | 60 | Gastric body | Low | T3N1M0 | IIIA | MSS | Adenocarcinoma | MSC3 | −3.085672185 |
| | | | near cardia | differentiation | | | | | | |
| T24 | Male | 44 | Lesser curvature | Low | T3N2M0 | IIIA | MSS | Adenocarcinoma | MSC2 | −2.57079003 |
| | | | of fundus of | differentiation | | | | | | |
| | | | stomach | | | | | | | |
| T25 | Male | 40 | Cardia, lesser | Middle-low | T4aN2M0 | IIIB | MSS | Adenocarcinoma | MSC3 | −1.248821673 |
| | | | curvature of | differentiation | | | | | | |
| | | | stomach, and | | | | | | | |
| | | | anterior wall | | | | | | | |
| | | | of stomach | | | | | | | |
| T26 | Female | 63 | Gastroesophageal | Middle-low | T4aN3aM0 | IIIC | MSS | Adenocarcinoma | MSC3 | −0.663776766 |
| | | | junction | differentiation | | | | | | |
| T27 | Male | 76 | Lesser curvature | Middle-low | T3N2M0 | IIIA | MSS | Adenocarcinoma | MSC1 | 3.338065613 |
| | | | of cardia | differentiation | | | | | | |
| T28 | Female | 54 | Gastric antrum | Low | T4bN3aM0 | IIIC | MSS | Adenocarcinoma | MSC1 | 3.348826952 |
| | | | | differentiation | | | | | | |
| T29 | Female | 57 | Gastric antrum | Middle | T3N0M0 | IIA | MSS | Adenocarcinoma | MSC3 | −3.695337416 |
| | | | | differentiation | | | | | | |
| T30 | Male | 30 | Lesser curvature | Low | T1N1M0 | IB | MSS | Adenocarcinoma | MSC3 | −5.25894177 |
| | | | of stomach | differentiation | | | | | | |
| T31 | Female | 67 | Gastric antrum | Low | T3N1M0 | IIB | MSS | Adenocarcinoma | MSC1 | 2.943184241 |
| | | | | differentiation | | | | | | |
| T32 | Female | 61 | Gastric antrum | Low | T4aN3bM0 | IIIC | MSS | Adenocarcinoma | MSC3 | −3.135943899 |
| | | | | differentiation | | | | | | |
| T33 | Male | 52 | Gastric angle | Middle | T3bN1M0 | IIB | MSS | Adenocarcinoma | MSC2 | 3.85760808 |
| | | | | differentiation | | | | | | |

The specific clinical and molecular features of GC samples are shown in Table 1.

Table 1 Clinical and molecular features of GC samples from Shandong provincial hospital

Transcriptomic and metabolomic sequencing and analysis

### Library Preparation for Transcriptome Sequencing

RNA integrity in the SDPH-GC cohort was assessed using RNA Nano 6000 Assay Kit on a Bioanalyzer 2100 system (Agilent Technologies, CA, USA). RNA samples were prepared using a total of 1 μg of RNA for each sample as input material. Finally, polymerase chain reaction (PCR) products were purified (AMPure XP system) and library quality was assessed on an Agilent Bioanalyzer 2100 system. Clustering of coded samples was performed on a cBot Cluster Generation System using the TruSeq PE Cluster Kit v3-cBot-HS (Illumia) according to the manufacturer's instructions. After cluster generation, library preparation was sequenced using the Illumina Novaseq platform to generate 150 bp paired-end reads.

### Transcriptome Data Analysis

In SDPH, raw data in fastq format (raw reads) was first processed by an internal per1 script. In this step, clean data (clean reads) was obtained by removing reads1 containing adapter and poly-N, and low quality reads from the raw data. All downstream analyses were based on clean data with high quality. Indexes of reference genomes were created using Hisat2 v2. 0. 5, and paired-end clean reads were mapped to the reference genomes using Hisat2 v2. 0. 5. The number of reads mapped to each gene was calculated using featureCounts v1. 5. 0-p3. Fragments per kilobase million (FPKM) was calculated for each gene on the basis of the length of the gene and the number of reads mapped to that gene.

### Liquid Chromatography-Mass Spectrometry (LC-MS)/MS of Metabolome

Analysis was performed using a Vanquish UHPLC (Thermo) coupled to an Orbitrap (Q Exactive HF-X/Q Exactive HF). Hydrophilic interaction liquid chromatography (HILIC) isolation was analyzed using a 2. 1 mm×100 mm ACQUIY UPLC BEH Amide 1.7 Um column (Waters, Ireland). In electrospray ionization (ESI) positive-negative mode, a mobile phase contained A=25 mM ammonium acetate and 25 mM ammonium hydroxide in water and B=acetonitrile. A gradient was 98% B for 1. 5 min and linearly decreased to 2% over 10.5 min, then held for 2 min before being increased to 98% over 0. 1 min, and a 3 min re-equilibration time was used. In automated MS/MS acquisition, an instrument was set within an m/z range of 70-1200 Da for acquisition, a resolution was set to 30000, and an accumulation time was set to 50 ms and exclusion was performed within an exclusion time of 4 s.

### Metabolome Data Processing

Raw MS data was converted to MzXML files using ProteoWizard MSConvert and then imported into XCMS software. Isotopes and adducts were annotated using the algorithm collection MEtabolite pRofile Annotation (CAMERA). Only variables with 50% non-zero measurements in at least one group were retained in the extracted ionicfeatures. Identification of compounds of metabolites was performed by comparing precise m/z values (<10 ppm) with MS/MS spectra of an internal database with available authentic standards.

### Collection and Collation of Metabolic Gene Features

Metabolic gene features were collected and extracted from previous studies. Briefly, eight gene sets of metabolic super-pathways, including metabolic genes of amino acid (348), carbohydrate (286), energy (110), glycan (247), lipid (766), nucleotide (90), TCA, (148), and vitamin cofactor (168), are collated according to the recent annotations from the Reactome, a biomolecular pathways knowledge base. Networks of collected MPIs (1870 metabolites and 4132 proteins) were utilized, and the linkage between metabolite-interacting genes and four or more networks of MPIs were considered as an effective metabolite subgroup.

### Consensus Molecular Clustering of Metabolic Marker Genes

Consensus unsupervised clustering was performed using the NMF algorithm to identify different metabolic patterns on the basis of 456 metabolic features formed by 456 genes. A metabolic gene profile was decomposed into two non-negative matrices W and H (i.e., A≈WH). The matrix A was subjected to repeated factorization and outputs thereof were subjected to aggregation to obtain consensus clusters of GC samples. The optimal number of clusters was selected according to a correlation coefficient, an RSS coefficient, a dispersion coefficient and a silhouette coefficient. Consensus clustering was performed in GEO, TCGA, SDPH and cancer cell line encyclopedia (CCLE) databases using R software package “NMF” (version 0. 22.0) with Brunet algorithm and 60 nruns.

### GSVA and Gene Ontology (GO) Annotation

A GSVA algorithm and an R package “GSVA v1. 38.2” were used to study the variation of biological processes in different MSC subtypes. Well-defined biological features were obtained from the Hallmark and KEGG gene sets (downloaded from the MSigDB database v7. 1), Thorsson et al. constructed a gene set, and Zeng et al. collected an immune-tumor pathway and a self-constructed cuproptosis gene set.

### Construction of MSPG Marker Scoring System

An MSPG scoring scheme was constructed to quantify the relative metabolite levels of individual patients using PCA. Specifically, overlapped DEGs among the three MSC subtypes were determined, and prognostic analysis of each gene was performed using a univariate Cox regression model. Genes with significant prognostic significance (P<0. 05) were extracted for further feature selection using the VSURF v1. 1. 0 package in combination with the random forest algorithm, with the random tree set to 10000 as previously described. Expression profiles of the final determined 11 gene features were collated (TTC28, GPA33, PDE7B, SCN4B, LMNB2, HNF4G, LGALS3, SPARCL1, CDS1, ZNF532, and FRY) for PCA analysis, and the normalized dataset and feature vectors were subjected to matrix multiplication to obtain PC scores, and scores of PCs 1, 2, and 3 were summed up as a metabolic subtype prognostic gene set score. The method mainly focuses on the score of the set with the largest block of well-correlated (or inversely correlated) genes in the set, and reduces the contribution of genes that do not track other set members. MSPG-score was defined as: MSPG=Σ(PC1+PC2+PC3). For example, in 33 SDPH datasets, expression profile data of 11 gene features were screened for normalization transformation, followed by performing PCA using a Princomp package, with cor set to T, and other parameters set to default, and the obtained first three of PC scores of the samples were summed up to obtain MSPG-score for each sample. For example, the first three PCA scores of a T-01 sample were summed up: 2. 19458953+(−2. 889745548)+(−0. 287095177)=−0. 9822512.

### Collection of Transcriptomic and Clinical Information in ICI-Based Cohort

In a prospective phase 2 clinical trial (NCT #02589496), a metastatic GC cohort treated with pembrolizumab (PD-1 Ab) was included in the study, and gene expression profiles of biopsy samples prior to treatment were downloaded and collated for further analysis. In addition, two independent immunotherapy cohorts: metastatic melanoma treated with navulizumab/pembrolizumab (anti-pd-1 mcAb) and metastatic urothelium carcinoma (mUC) treated with atelizumab (anti-PD-L1 mcAb) were collected and utilized for external validation. According to the response evaluation criteria in solid tumors (RECIST) 1. 1 guidelines, the immune response rate was calculated as the percentage of patients with confirmed complete remission (CR) or partial remission (PR), while stable disease (SD) or progressive disease (PD) was considered non-responsive.

### Statistical Analysis

In this study, statistical analysis was performed using R-4. 0. 2. For quantitative data, statistical significance was estimated using student t-test for normally distributed variables and analyzed using Wilcoxon rank sum test for non-normally distributed variables. For comparison of more than two groups, Kruskal-Wallis test and one-way analysis of variance (ANOVA) were used as non-parametric and parametric methods, respectively. Chi-square test and Fisher exact test were used to analyze contingency tables at specific grouping conditions. Kaplan-Meier survival analysis and a Cox proportional hazard model were used to analyze the relationship between metabolic transcriptome patterns and prognosis. The samples were stratified into high and low MSPG-score subgroups using the survival-pointcut function in the “survival” package. The prognostic grading and the performance and AUC of an MSPG-score model were evaluated using a characteristic curve (ROC). All comparisons were two-sided, with a set to 0. 05, and multiple hypothesis testing was performed using the Benjamini-Hochberg method to control for false discovery rate (FDR).

### 2. Results

### 2. 1. The Reveal of Metabolic Dysregulation in GC Cells by Transcriptomic Analysis

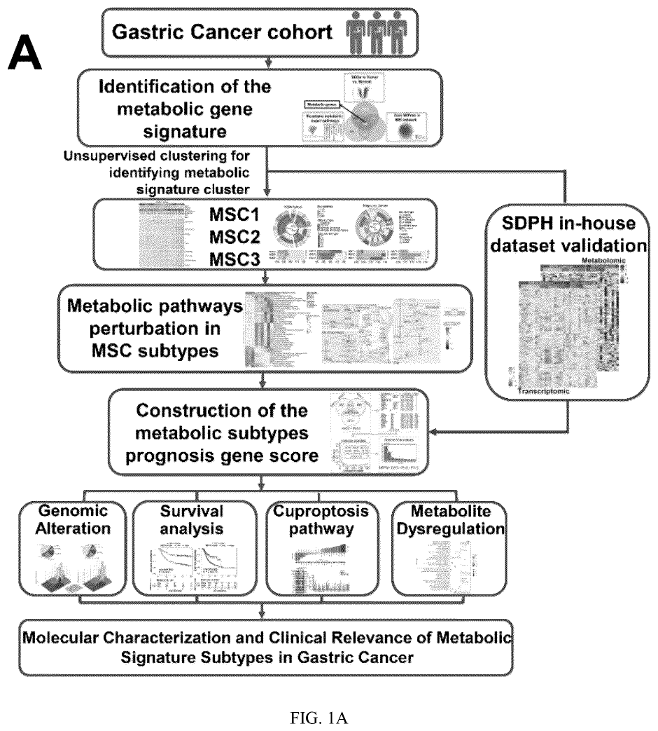

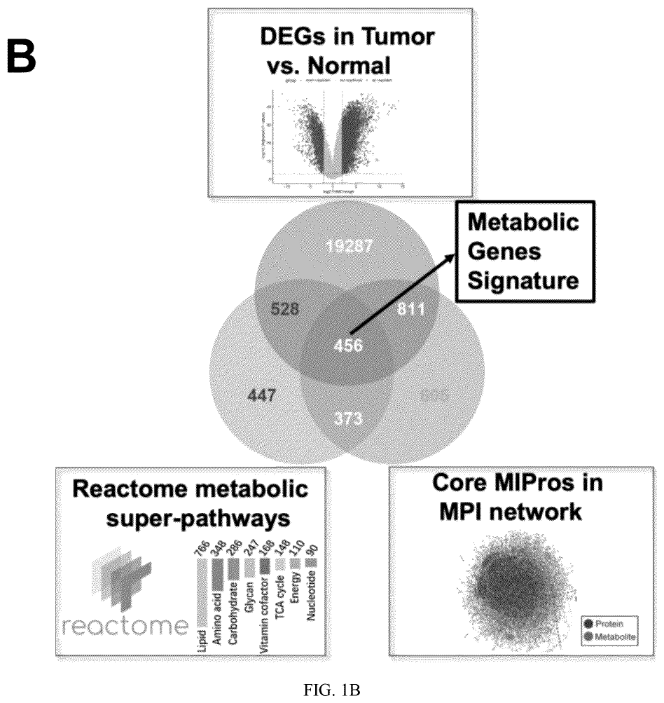

The flow of study design is first summarized to elucidate the construction scheme for metabolic markers and patterns for GC (FIG. 1A). To identify genes related to metabolic features of GC, core metabolite-interacting proteins (MIPros) proposed by Chen et al. are collected. In DEGs between gastric tumors and adjacent normal tissues, a gene set of metabolic super-pathways collated by Peng et al. is collected (Method section). A total of 456 genes exhibiting key metabolic features, recognized as GC metabolic marker genes, are represented in a WENN diagram (FIG. 1B).

### 2. 2. Metabolic Signature Clustering Analysis of GC Cells

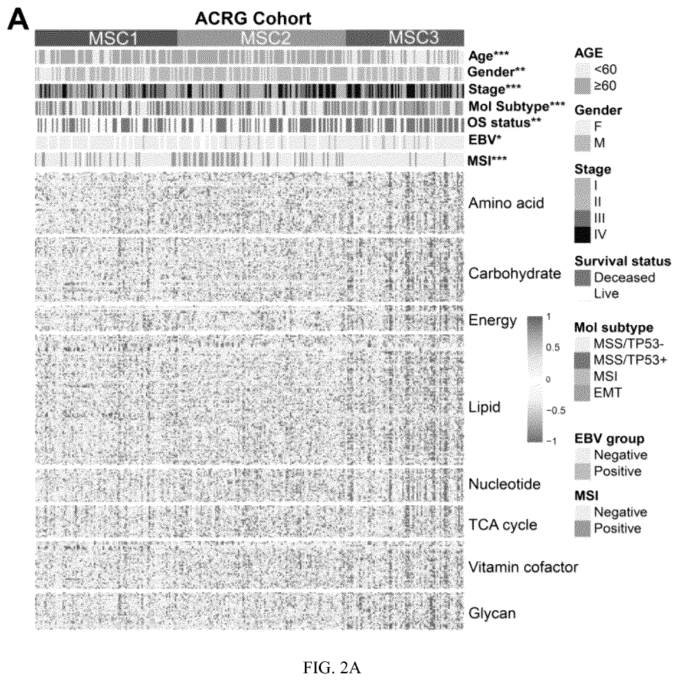

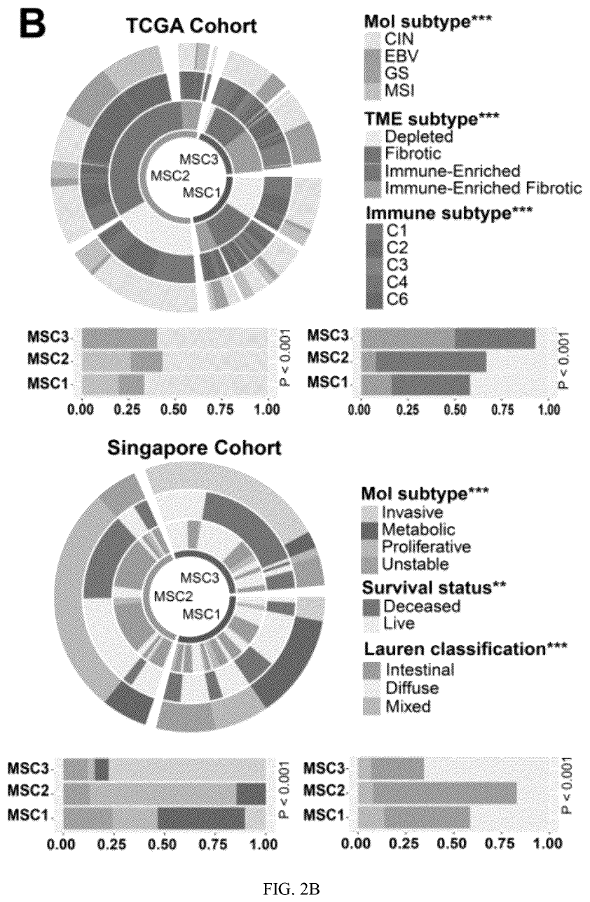

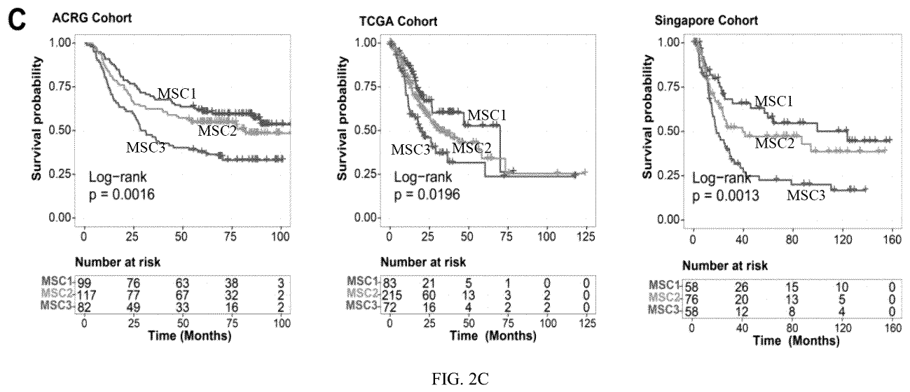

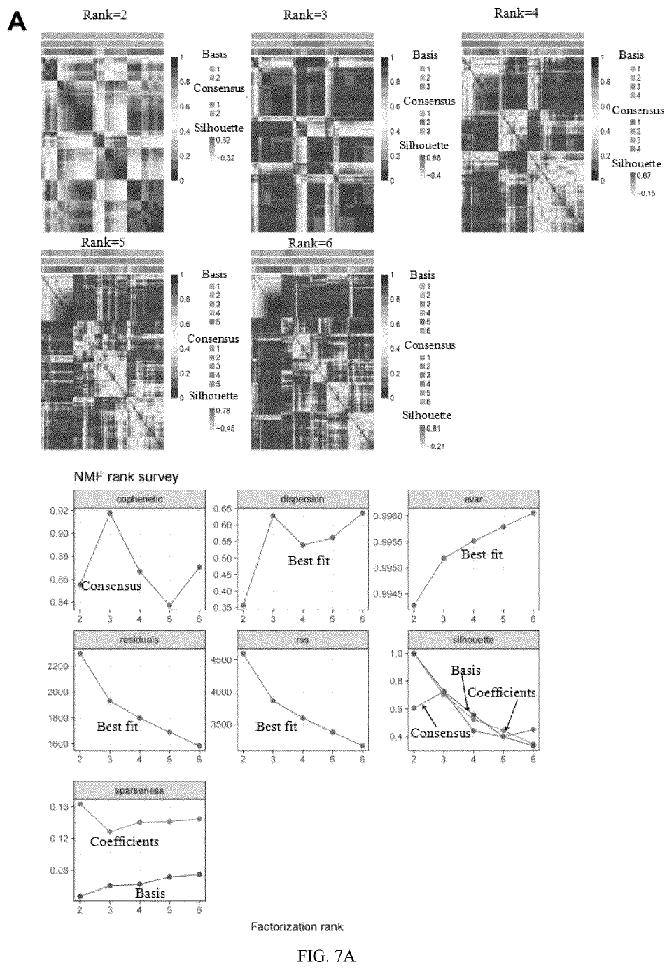

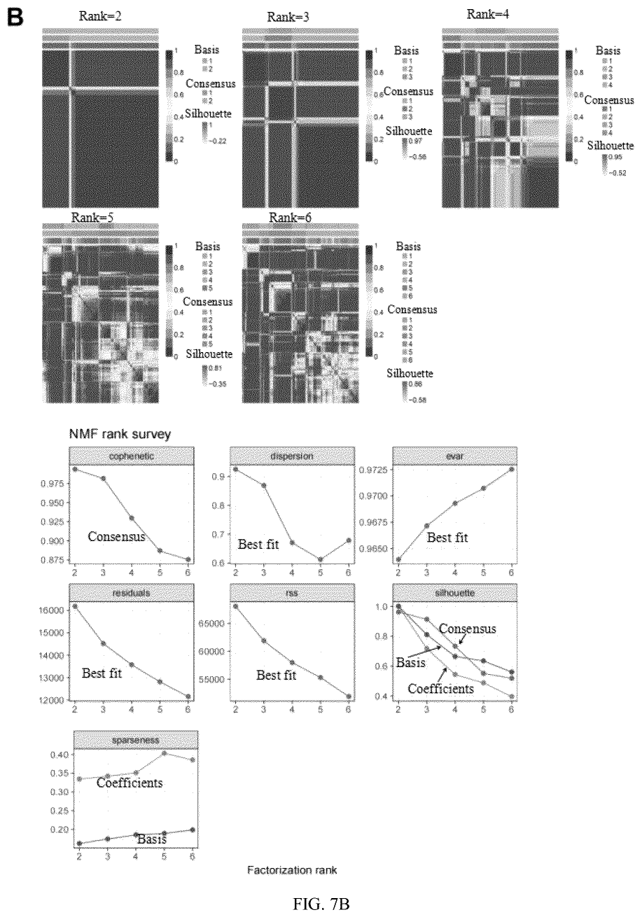

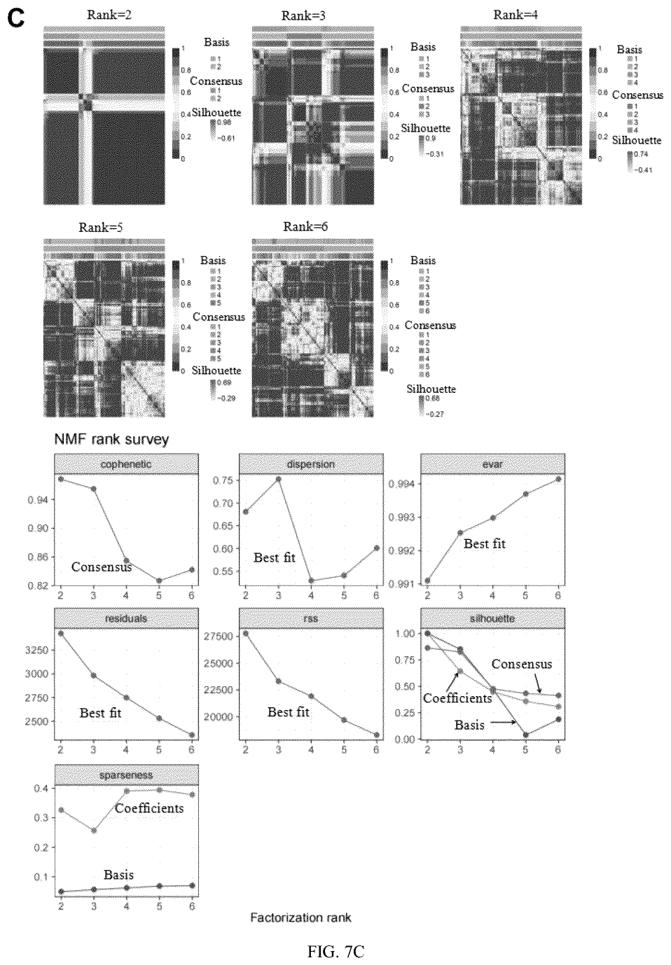

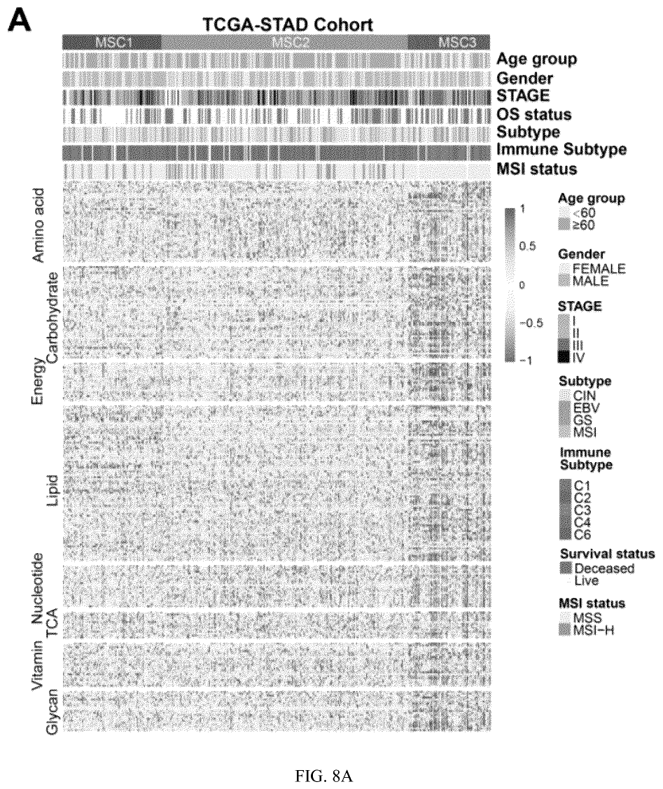

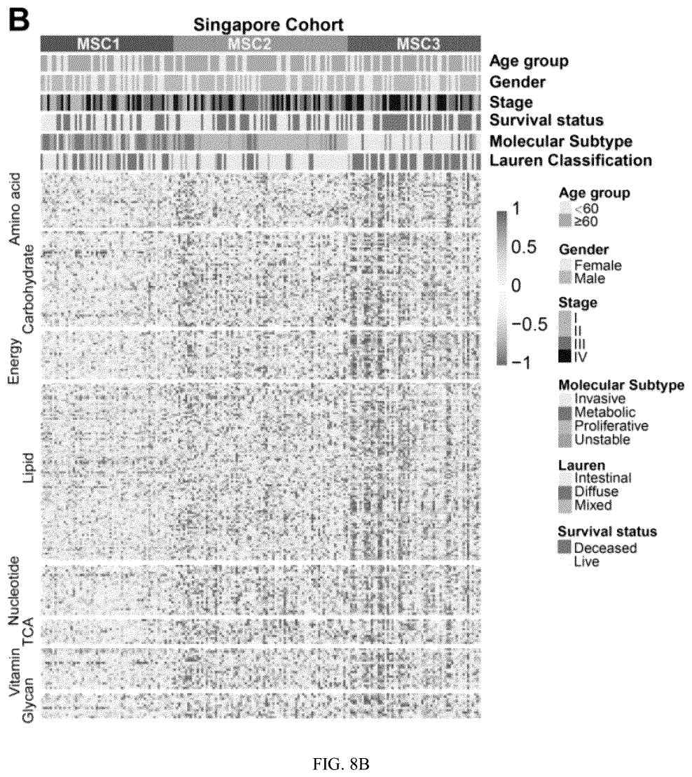

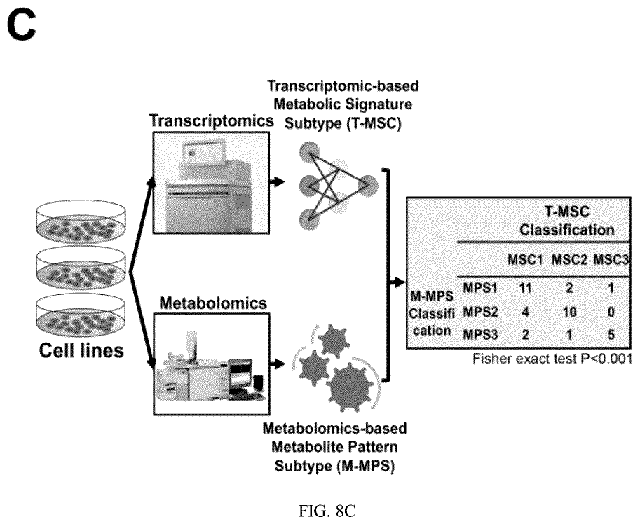

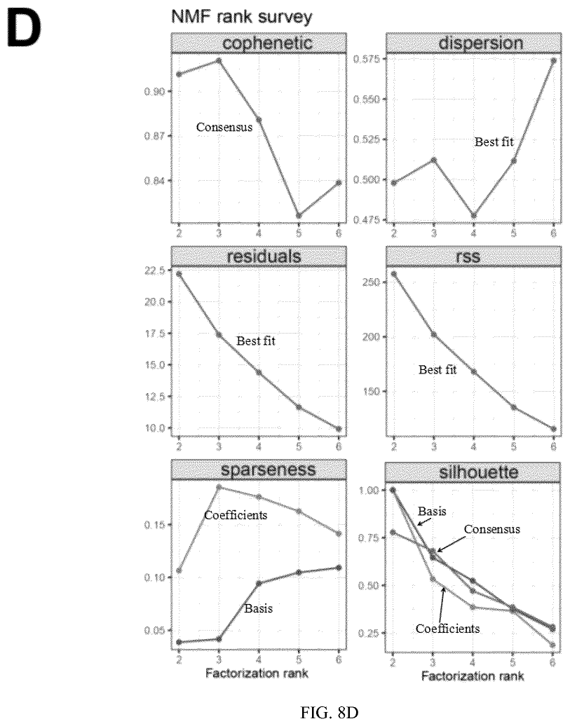

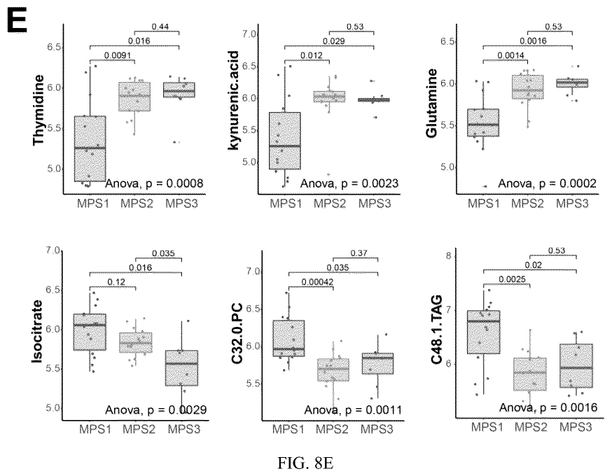

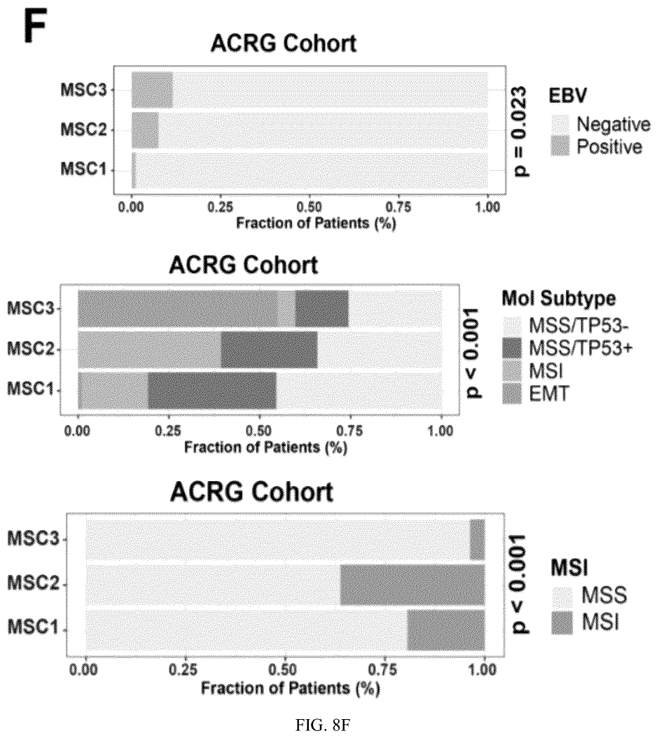

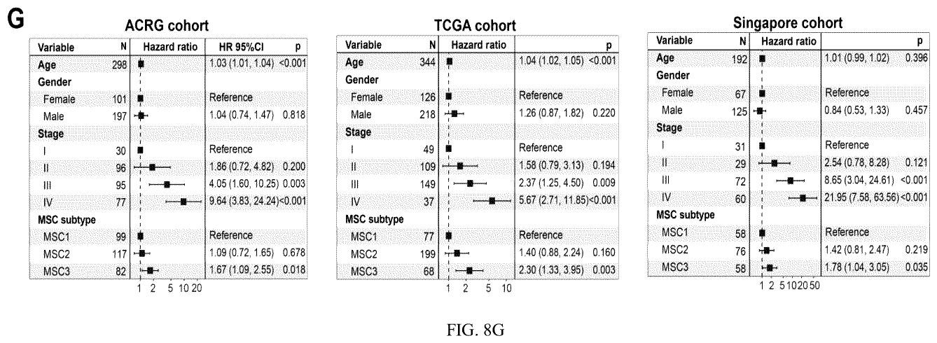

The above analysis shows the special metabolic network and features of GC. To reveal the metabolic heterogeneity of GC cells, metabolic gene signatures are used to cluster GC tumors into different subtypes. According to the cophenetic, RSS and dispersion parameters of the unsupervised consensus NMF clustering analysis (FIG. 7A-7C), three clusters are the optimal number of clusters {Analysis name: the unsupervised consensus NMF clustering metrics of cophenetic, RSS, and dispersion}. Subsequently, on the basis of metabolic signatures, GC tumors are classified into three clusters (MSC1, MSC2, and MSC3), and metabolic gene transcription profiles are produced according to metabolic super-pathways (FIG. 2A). MSC1 is predominantly designated as a mixed subtype, characterized by a relative up-regulation of TCA cycle and lipid metabolic pathways. MSC2 is considered as a nucleotide and amino acid subtype, characterized by a relative up-regulation of nucleotide and amino acid metabolic pathways. MSC3 is subsequently designated as a glycan subtype, characterized by a significant up-regulation of glycan, carbohydrate and energy metabolic pathways (FIG. 2A). The reproducibility of this metabolic clustering result is externally validated by expression profiles of TCGA and Singapore cohorts (FIG. 8A-8B). Previous studies have identified several molecular subtype nomenclatures for GC on the basis of transcriptomic and genomic analyses. Herein, the relationship between metabolic marker subtypes and previously identified molecular subtypes and clinical features is investigated (FIG. 2B, and FIG. 8F). Overall, MSC3 is predominantly associated with EMT subtypes having a poor prognosis in the ACRG group, genomically stable (GS) subtypes in the TCGA group, and infiltration subtypes in the Singapore group, respectively. The EMT, GS, and infiltration subtypes have the worst prognosis and fewer mutations. The MSC2 subtype has a high proportion of intestinal tissue subtypes, enriched MSI tumors and accompanied by mutations. In addition, the prognostic analysis shows significant differences among the three MSC subtypes, with MSC3 exhibiting shorter survival time, while MSC1 and MSC2 exhibiting longer survival times in ACRG, TCGA and Singapore groups (P<0.05, log-rank test, FIG. 2C). Multivariate Cox proportional hazards regression analysis (MCRA) further demonstrates that the metabolic signature clustering model is associated with patient's survival outcomes after clinicopathological factors in the 3 groups are adjusted (FIG. 8G). Overall, the results suggest that GC cells can be classified into three metabolic phenotypes that are characterized by different metabolic features and survival outcomes. Notably, the up-regulation of the metabolism of glycan, carbohydrate and energy is associated with aggressiveness of tumors.

### 2. 3. Metabolic Subtypes of GCs Characterized by Specific Clinical Features and Molecular Processes

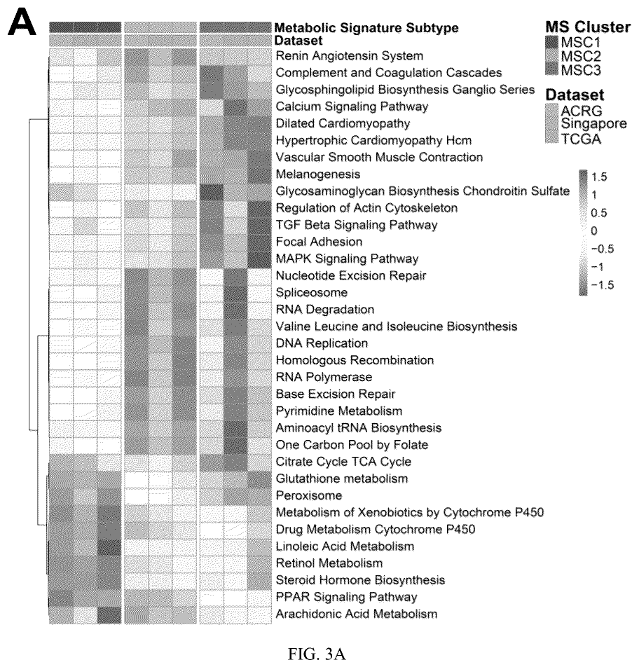

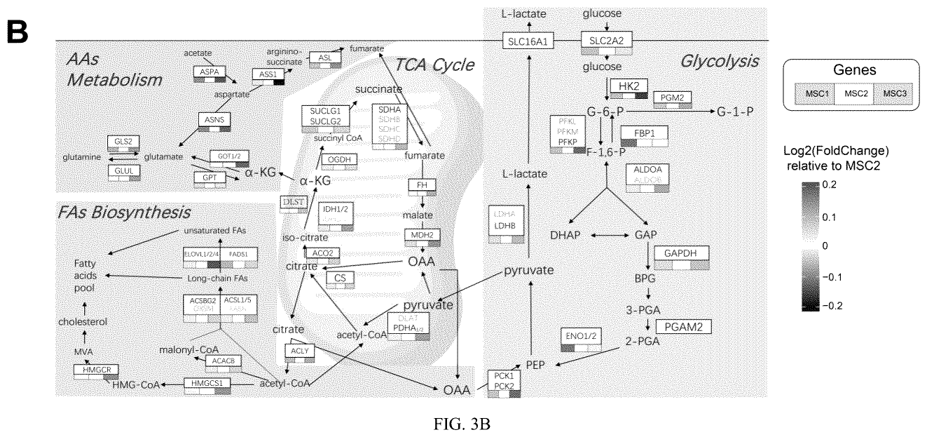

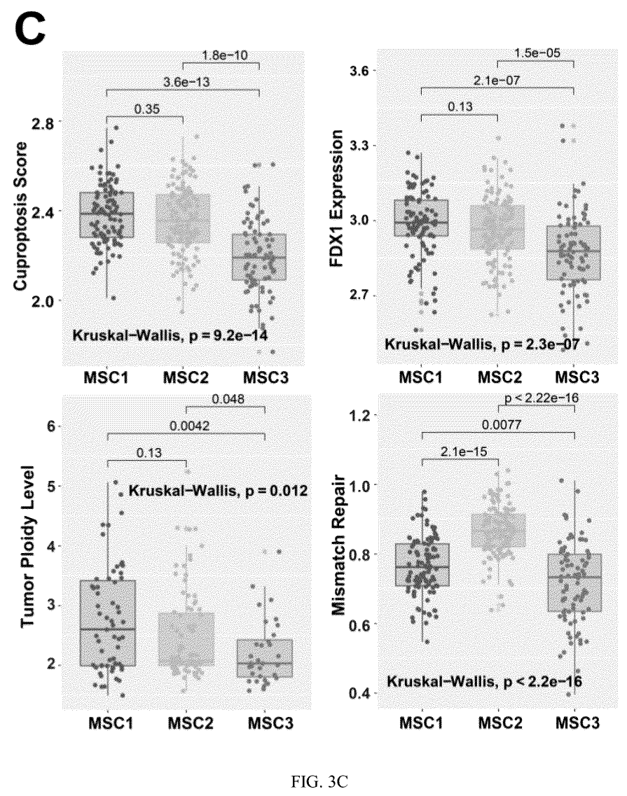

It is also determined whether mRNA expression of metabolic enzymes and corresponding metabolite abundance are correlated with the enrichment scores of metabolic pathways in different subtypes. Tumors within the MSC1 subtype are enriched for expression of genes involved in TCA cycle, including succinate dehydrogenase complex flavoprotein subunits (SDHA/B/C), succinate-CoA ligase GDP/ADP forming subunits (SUCLG1/2), and isocitrate dehydrogenase (IDH1/2) (FIG. 3B). In contrast, the expression of metabolic enzymes involved in glycolysis is relatively elevated in MSC3. For example, the expression of hexokinase 3 (HK3), 6-phosphofructose-2 kinase/fructose-2, 6-bisphosphatase 3 (PFKFB3), lactate dehydrogenase (LDHA/B), and enolase (EN01/2) is higher in MSC3 than in other subtypes. It is also noted that amino acid metabolism-related molecules, glutaminase 2 (GLS2), and asparagine synthetase (ASNS) are up-regulated in MCS2 of GCs (FIG. 3B).

Considering that cuproptosis-related TCA cycle and pyruvate metabolism are significantly dysregulated in different metabolic clusters, the levels of cuproptosis metabolic pathways and core molecule FDX1 in different MSC subtypes are further compared. As a result, cuproptosis and FDX1 are significantly reduced in MSC3 on ACRG, TCGA and Singapore datasets (FIG. 3C). In addition, genomic instability markers of tumor ploidy, somatic copy number alteration (SCNA), mutation load, and MMR are compared, and it is found that the level of instability markers of the MSC3 subtype is lower, while that of the MSC2 subtype is slightly higher (FIG. 3C).

### 2. 4. Construction of MSPG-Score and its Relevance Explored at Clinical and Molecular Level

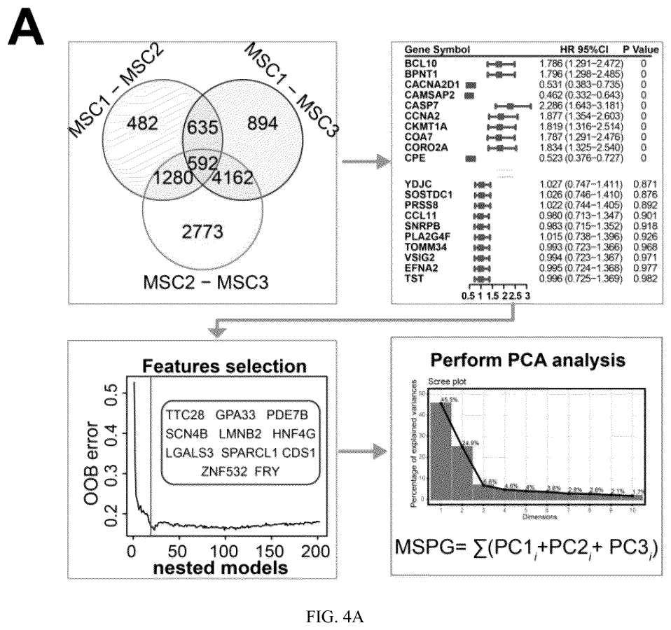

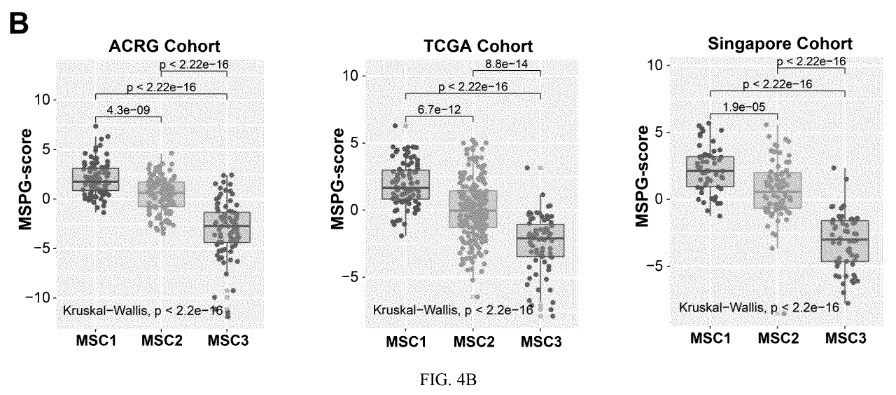

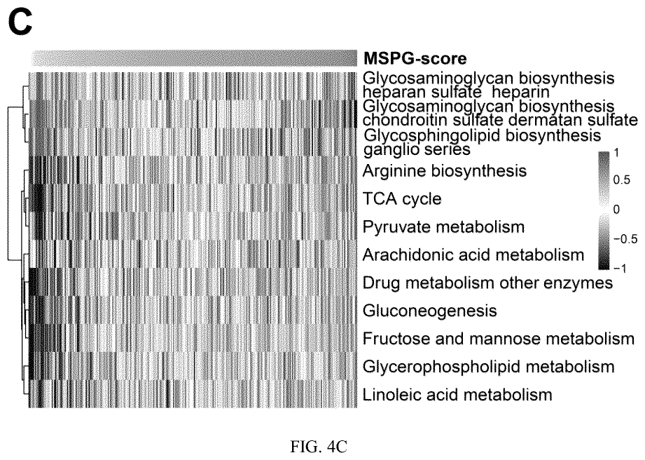

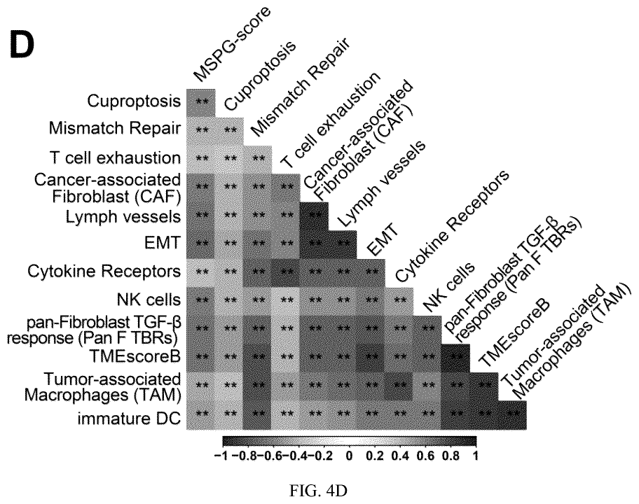

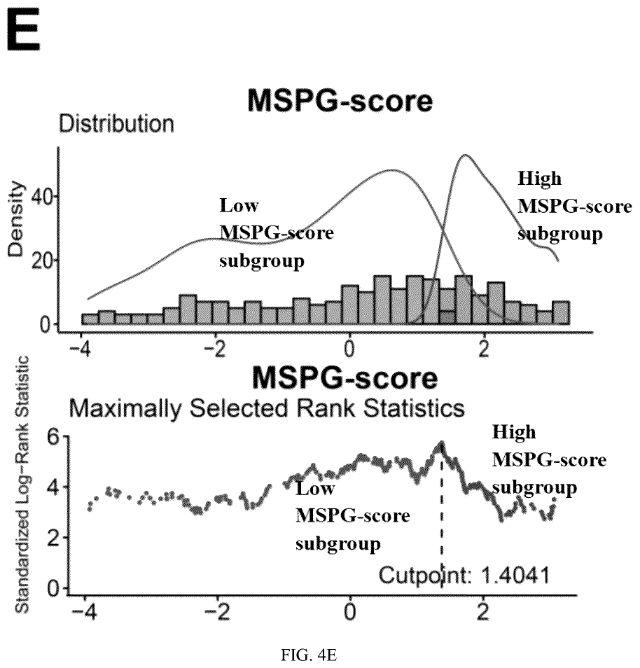

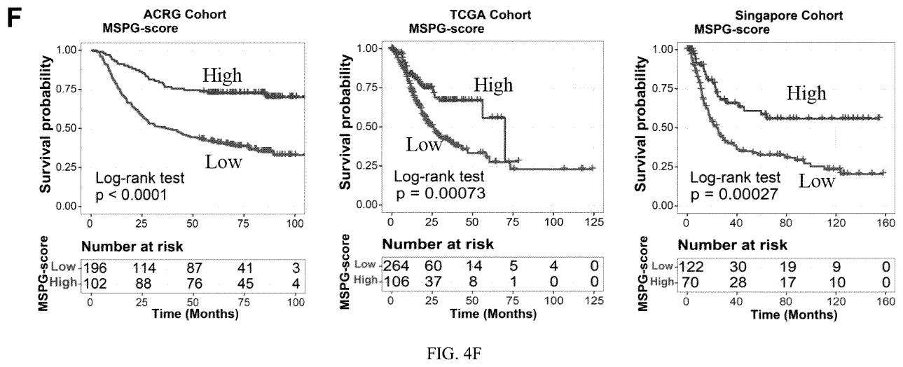

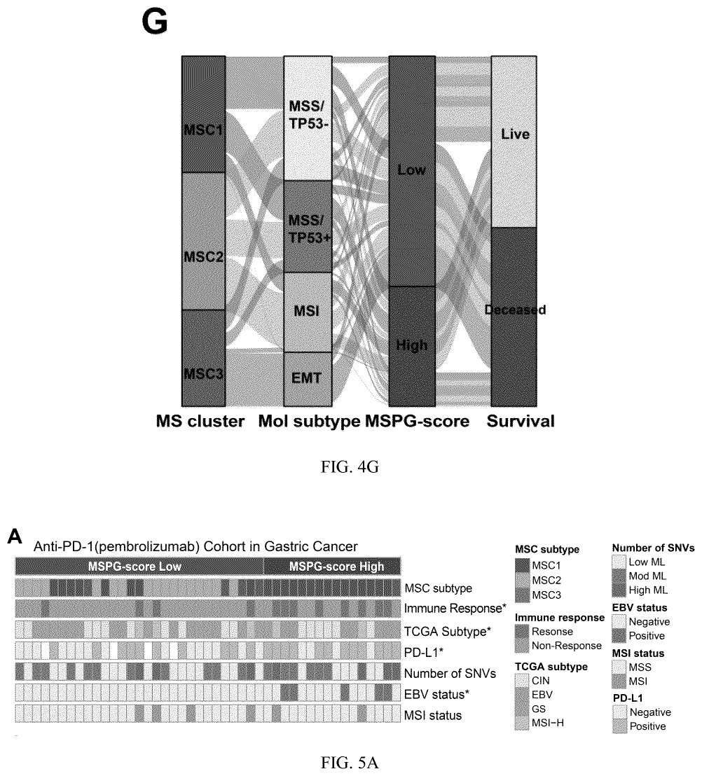

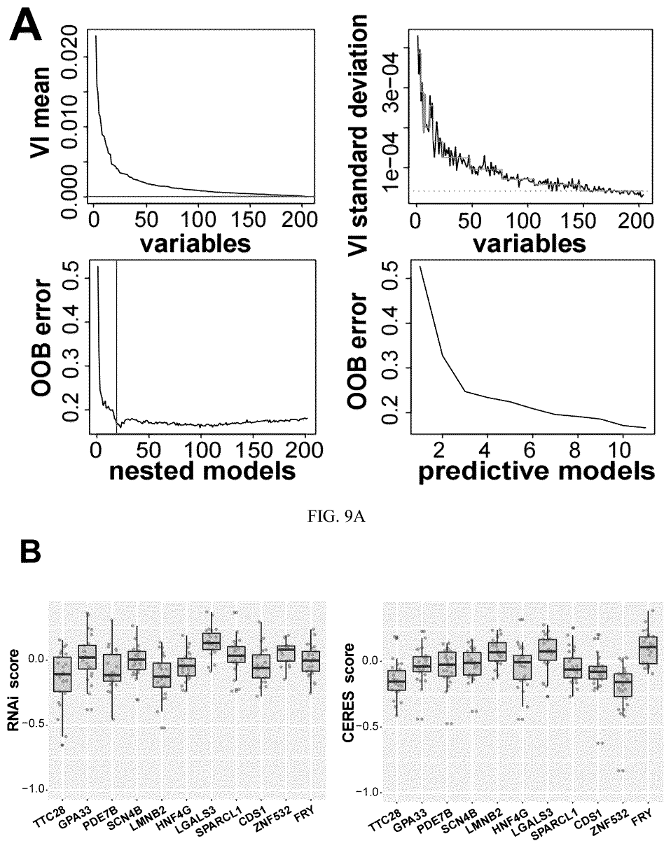

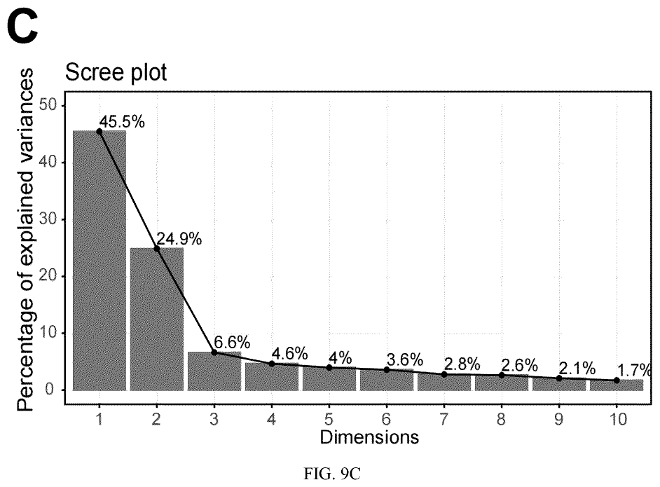

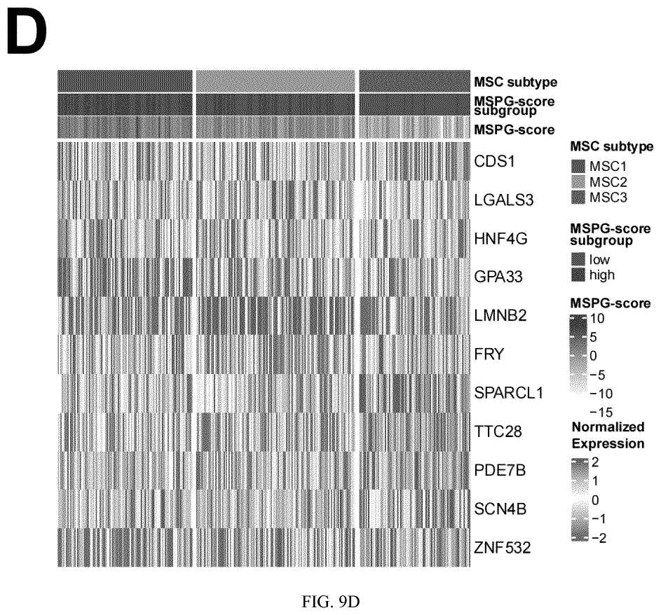

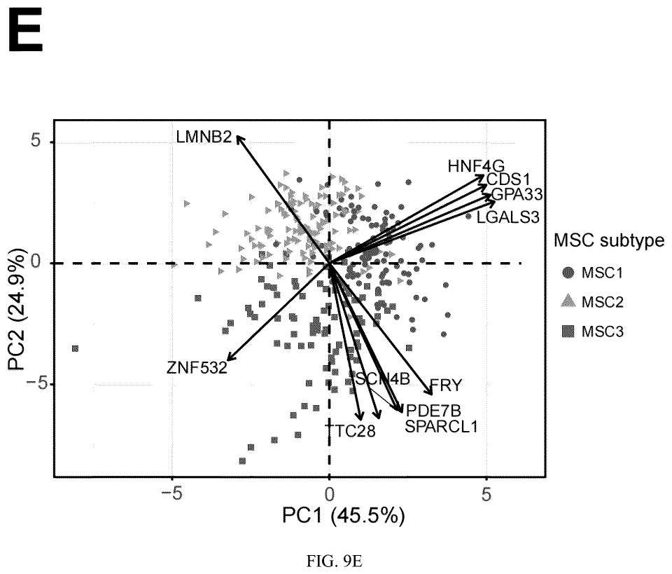

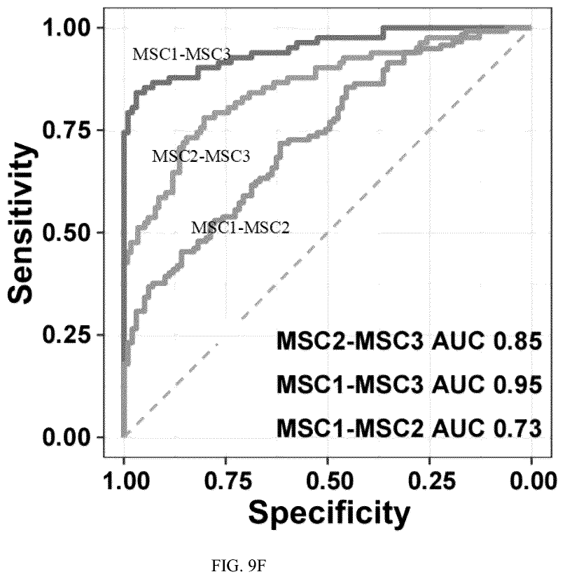

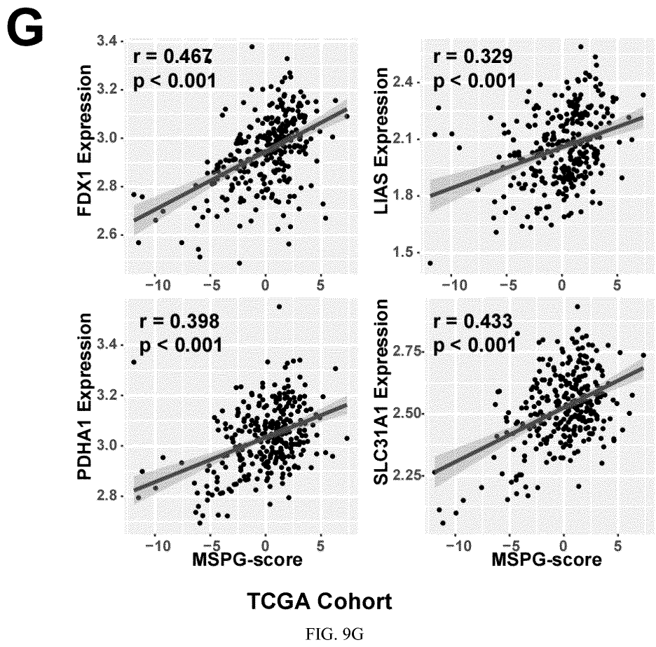

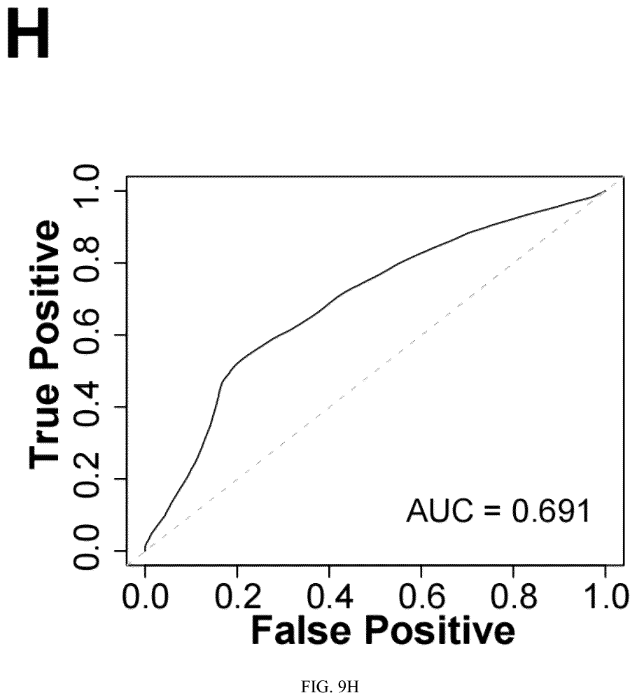

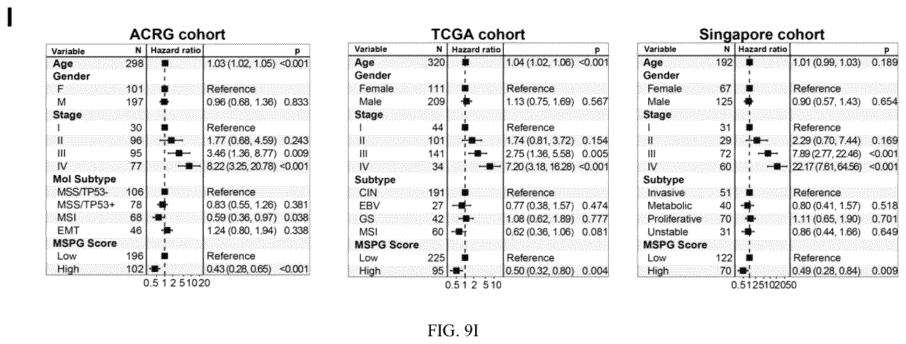

Briefly, first, the empirical Bayes algorithm is used to identify overlapped DEGs among the three MSC subtypes. 592 DEGs are obtained for univariate survival analysis to identify prognosis-related genes (Cox model, P<0. 05). The random forest algorithm is used for variable selection to further screen for gene feature map (FIG. 9A). Finally, 11 specific MSC-related prognostic genes are determined as the most discriminative gene features, including TTC28, GPA33, PDE7B, SCN4B, LMNB2, HNF4G, LGALS3, SPARCL1, CDS1, ZNF532, and FRY (FIG. 4A). In addition, RNAi score and CERES score reflect the cell viability effect after knockdown of a particular gene, and the results show that TTC28, HNF4G, and CDS1 have lower average scores in two screening systems (FIG. 9B). Moreover, the MSPG-score model (method section) scoring scheme is established using PCA method to quantify the metabolic patterns of individual tumors on the basis of the weighted sum of the first three PC scores. The first three PCs explain 77% of the variation (FIG. 9C). The heat map shows that CDS1, LGALS3, HNF4G, and GPA33 are predominantly expressed in the MSC1 subtype (FIG. 9D) and clustered together in the upper right quartile of the PC variable correlation plot (FIG. 9E). Thus, positive weighting coefficients of the PCs in MSC1 overexpressed genes result in higher weighted sum scores for the MSC1 subtype. Consequently, the MSC1 subtype exhibits the highest MSPG-score, followed by MSC2 and MSC3 (FIG. 4B). The multi-class AUC and pairwise comparison AUC are calculated to assess the performance of the MSPG-score in classifying the three MSC subtypes. The overall AUC value is 0.8454, which has a good diagnostic value for distinguishing the three subtypes. MSPG-score has the optimal ability to differentiate MSC1 from MSC3 (AUC=0. 9496), followed by MSC2 from MSC3 (0.8546), and MSC1 from MSC2 (0. 7321) (FIG. 9F).

To explore the potential biological mechanisms underlying MSPG-score, GSVA analysis is performed, and it is found that MSPG-score subtypes are significantly negatively correlated with glycan-related circuits, including glycosaminoglycan biosynthesis-heparin acetylheparan sulfate, glycosaminoglycan biosynthesis-chondroitin skin sulfate sulfate, and glycosphingolipid biosynthesis ganglionic series, and are positively correlated with arginine biosynthesis, TCA cycle, pyruvate metabolism, mannose metabolism, and arachidonic acid metabolism (FIG. 4C). The relationship between the known biometric features and MSPG-score is analyzed by Spearman rank correlation. A heat map of correlation matrix shows that MSPG-score is negatively correlated with EMT, transforming growth factor (TGF)-β response, T-cell depletion, inflammation-associated natural killer (NK) cells, and immature dendritic cells (DCs), and is positively correlated with cuproptosis and MMR (FIG. 4D). The expression of cuproptosis-reated core molecules (including FDX1, LIAS, PDHA1, and SLC31A1) is significantly and positively correlated with MSPG-score (FIG. 9G).

On the basis of the optimal cutpoint of ACRG cohort, the GC tumors are further classified into high and low MSPG-score subgroups (method section, FIG. 4E). Kaplan-Meier plot shows that the high MSPG-score subgroup has a significantly better prognosis (HR, 0.33 [95% CI, 0. 22-0. 50], P<0. 0001, FIG. 4F). This cutpoint value is also used in the TCGA group and the Singapore dataset, and these tumors are classified into high and low subgroups likewise. In two independent cohorts, patients with higher MPSG-score have a significant survival advantage (HR, 0.49 [95% CI, 0.32-0. 75], P=0. 0007; 0. 42 [95% CI, 0. 26-0. 68], P=0. 0003, FIG. 4F). The analysis results of the ROC curve also validate that the established scoring model has a predictive advantage in terms of prognostic estimation (AUC=0. 691, FIG. 9H). The multifactor Cox regression model analysis, which takes into account patients' age, gender and tumor stage, confirms that MSPG-score can serve as an independent prognostic biomarker for assessing the prognosis of patients with GC (FIG. 91-9K). Considering the complex association between MSPG-score and previous molecular subtypes, the workflow of the construction of MSPG-score is summarized using an alluvial plot (FIG. 4G). These results suggest that MSC3 with EMT subtypes is associated with lower MSPG-score.

### 2. 5. The Application of MSPG-Score Model in Anti-PD-1 Immunotherapy

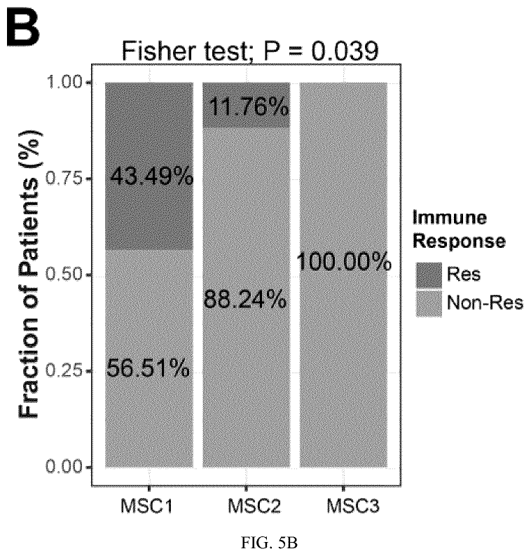

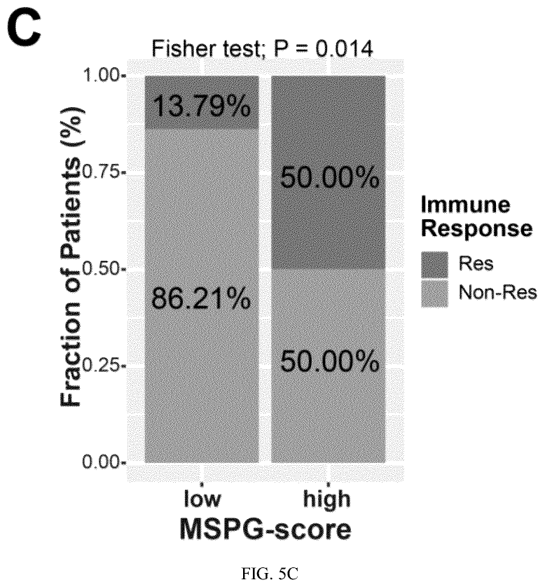

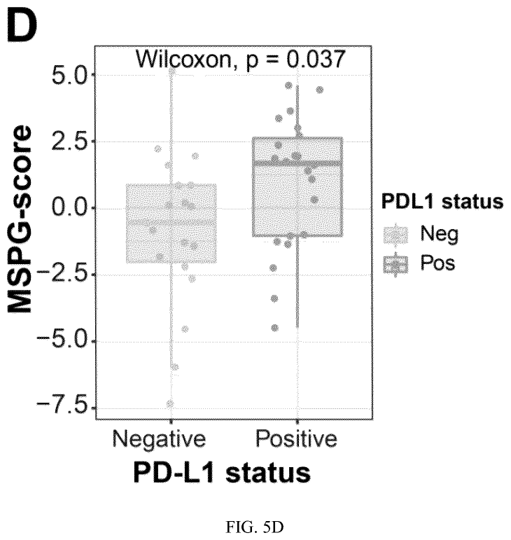

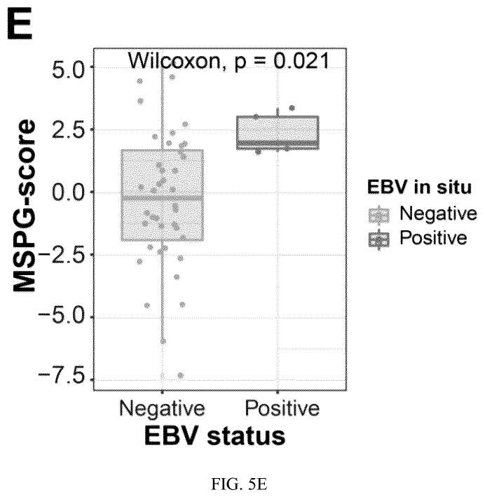

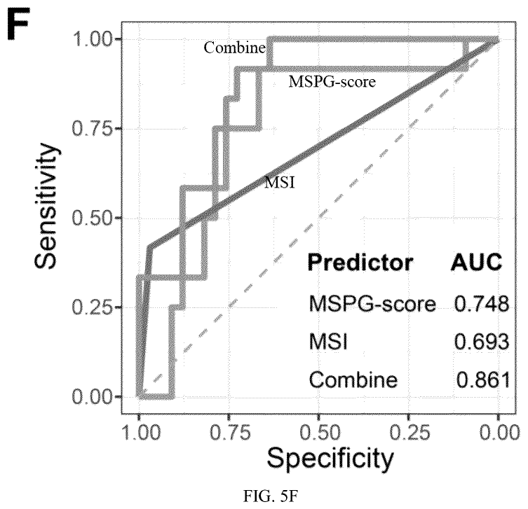

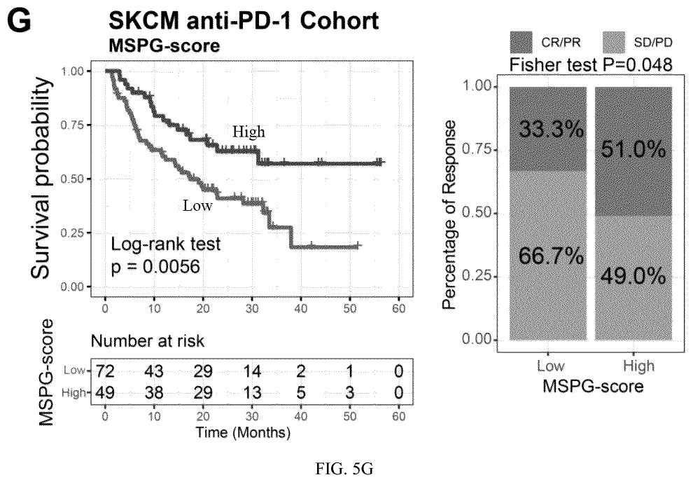

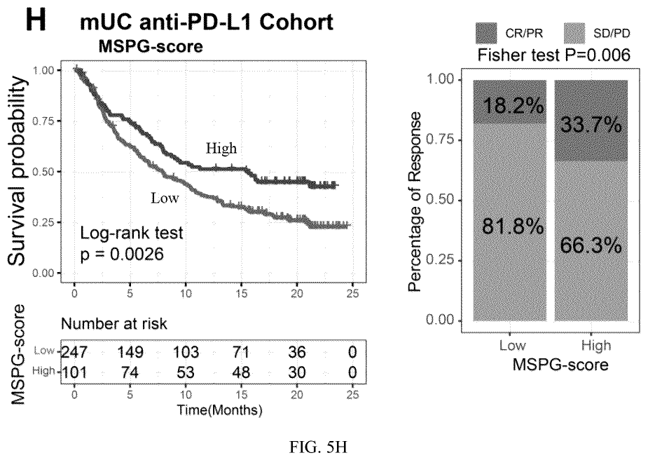

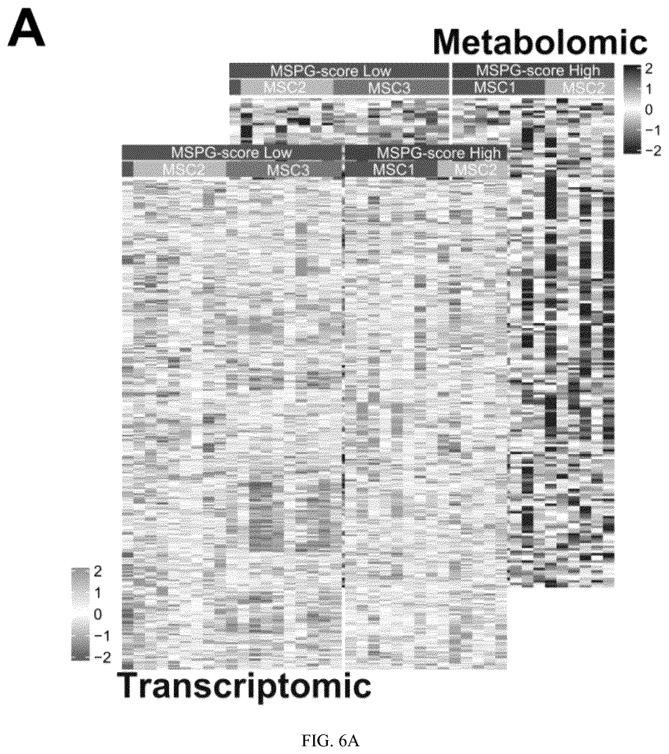

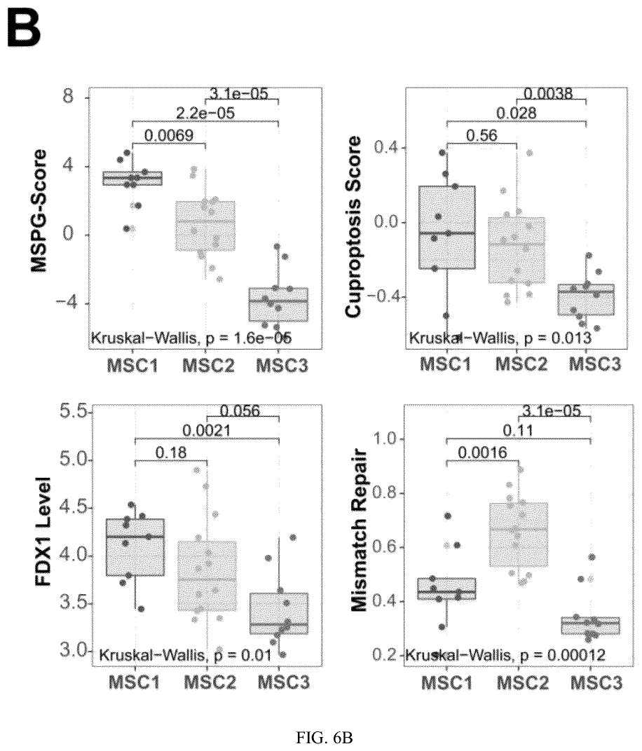

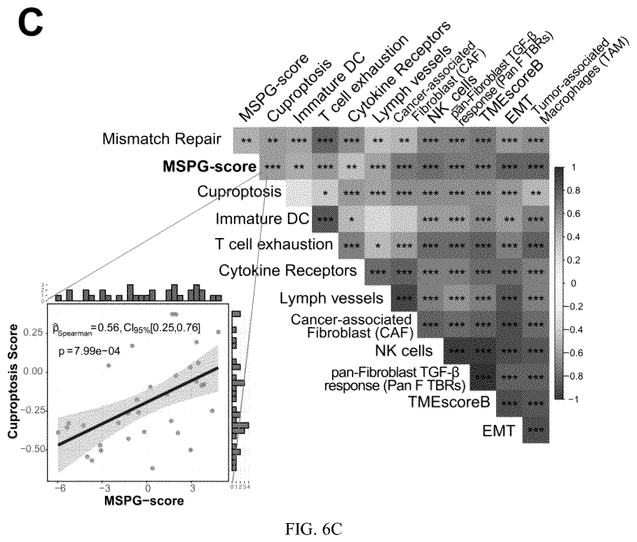

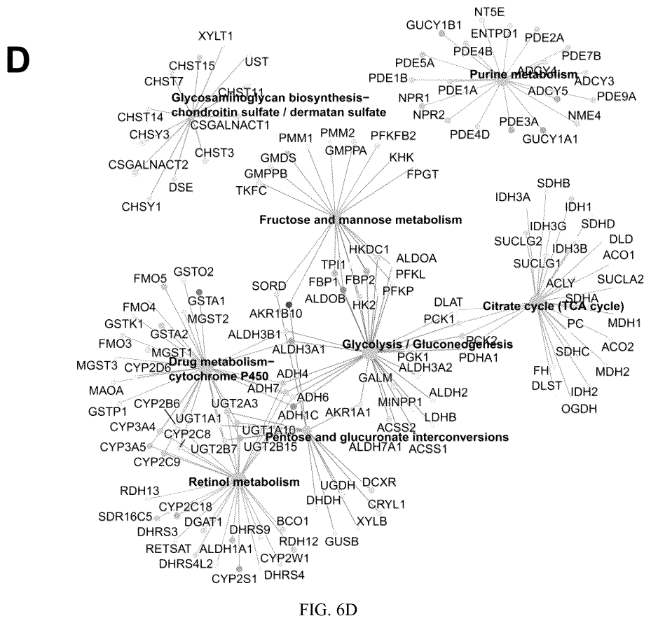

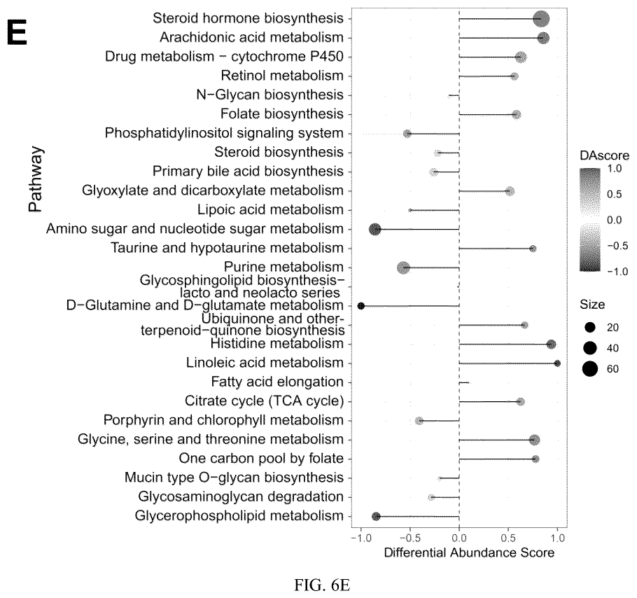

It is investigated whether MSC subtypes and MSPG-score can predict response to anti-PD-1 therapy in patients with GC. Significant therapeutic effects and immune responses are observed in patients with high MSPG-score (high vs. low, response rate: 50. 0% vs. 13. 8%) and MSC1/MSC2 subtypes (MSC1 vs. MSC2 vs. MSC3, response rate: 43. 5% vs. 11.8% vs. 0%) in the Pembrolizumab immunotherapy group (FIG. 6A-6C). Higher MSPG-score is found in PD-L1-positive, EBV-positive and immunotherapy-responsive samples (FIG. 5D-5E). The distribution pattern of MSPG-score across different MSC subtypes, TCGA molecular subtypes and PD-L1 status groups reveals that patients with higher MSPG-score are more likely to benefit from immunotherapy (Kruskal-Wallis H test, all P<0. 05). It is found that the combined model has a higher AUC in predicting immune response to ICI therapy compared to MPSG score and MSI alone (FIG. 5F).

In the melanoma group, patients in the high MSPG-score group demonstrate significant therapeutic benefit and immune response compared with the low MSPG-score group (HR, 0. 47 [95% CI, 0. 27 to 0. 81], P=0. 006; response rate: 51. 0% vs. 33. 3%, FIG. 5G). Consistent results are also observed in mUC cohort (HR, 0.63 [95% CI, 0. 46 to 0.85], P=0.003; response rate: 33. 7% vs. 18. 2%, FIG. 5H).

### 2. 6. Validation of Metabolic Features in an In-House Dataset

To further validate the metabolomics underlying the metabolic feature clustering and metabolic subtype scoring, paired transcriptomic and metabolomic analyses are performed on 33 frozen GC samples from the in-house SDPH group. A total of 1643 metabolites (1058 positive and 585 negative) with different classifications are annotated by untargeted LC-MS/MS approach. Transcriptomic data is processed by standard processes and further analyzed using MSC clustering and MSPG-score extraction methods. Consistently, three MSC subtypes and two MSPG-score subgroups are identified in SDPH-GC group (FIG. 6A). It is also found that MSC1 tumors have the highest MSPG-score, followed by MSC2 and MSC3 subtypes (FIG. 6B). Cuproptosis score and FDX1 expression are significantly down-regulated in the MSC3 subtype, while MMR-related scores are significantly enriched in the MSC2 subtype (FIG. 6B). A heat map of correlation matrix shows that MSPG-score is negatively correlated with EMT, TGF-B response, T-cell depletion, inflammation-associated NK cells, and immature DCs, but positively correlated with cuproptosis and MMR (FIG. 6C). The metabolic network module shows specific enrichment of metabolic pathways and metabolic genes between the two MSPG-score subgroups. Genes related to the TCA cycle, drug metabolism P450, fructose and mannitol metabolism, and retinol metabolism are significantly up-regulated in the high MSPG-score subgroup, while glycosaminoglycan biosynthesis-chondroitin sulfate/dermatan sulfate and purine metabolism are significantly increased in the low MSPG-score subgroup (FIG. 6D). In addition, KEGG metabolic pathway-based DA analyses are performed on the high and low MSPG-score subgroups to investigate the dysregulation of metabolic pathways. In this study, metabolites involved in glycan biosynthesis, amino sugar and nucleotide sugar metabolism, purine metabolism, glycerophospholipid metabolism, and fatty acid metabolism pathways exhibit enhanced DA scores in the low MSPG-score subgroup, while arachidonic acid metabolism, drug metabolism-cytochrome P450, histidine metabolism, and citrate cycle exhibit elevated DA scores in the high MSPG-score subgroup (FIG. 6E). Thus, the consistency of metabolic gene expression and metabolite abundance in differentiating MSPG-score subgroups is demonstrated.

1. A molecular subtyping method for gastric cancer (GC) based on metabolic genes, comprising the following steps:
S1, acquiring gene/protein expression profiling data and clinical information data of patients with GC;
S2, acquiring GC metabolism-related genes and GC metabolite-related features formed thereby on the basis of established studies; and
S3, performing consensus unsupervised clustering using a non-negative matrix factorization (NMF) algorithm, specifically, identifying different metabolic patterns on the basis of the GC metabolite-related features formed by the GC metabolism-related genes; decomposing a metabolic gene profile into two non-negative matrices, W and H; and repeating factorization of a matrix A, and aggregating outputs thereof, to obtain consensus clusters of GC samples, and classifying GC into three heterogeneous clusters with different metabolic features, prognoses, proteogenomic variants and metabolites, i.e. three molecular subtypes, named metabolic signature cluster (MSC) 1, MSC2 and MSC3.

2. The molecular subtyping method for GC according to claim 1, wherein in step S1, sources of the gene/protein expression profiling data and clinical information data of patients with GC comprise national center of biotechnology information-gene expression omnibus (NCBI-GEO), the cancer genome atlas (TCGA), clinical proteomic tumor analysis consortium (CPTAC), and actual clinical samples.

3. The molecular subtyping method for GC according to claim 1, wherein the specific method for step S2 comprises:
collating a gene set of metabolic super-pathways, including metabolic genes of amino acid, carbohydrate, energy, glycan, lipid, nucleotide, tricarboxylic acid (TCA), and vitamin cofactor, according to the annotation of a biomolecular pathways knowledge base; utilizing networks of collected metabolite-protein interactions (MPIs) and considering the linkage between metabolite-interacting genes and four or more networks of MPIs as an effective metabolite subgroup; and performing differential expression analysis between TCGA tumors and adjacent normal tissues for overlapped genes in a dataset, and finally identifying GC metabolite-related features formed by a plurality of metabolic genes.

4. The molecular subtyping method for GC according to claim 1, wherein the three heterogeneous clusters are determined according to a correlation coefficient, a residual sum of squares (RSS) coefficient, a dispersion coefficient and a silhouette coefficient.

5. A construction method for a GC prognostic model based on a molecular subtyping method for GC according to claim 1, comprising the following steps:
S1, acquiring overlapped differentially expressed genes (DEGs) among the three MSC subtypes on the basis of a subtyping method, and performing prognostic analysis of each gene using a univariate Cox regression model;
S2, extracting genes with significant prognostic significance using a random forest algorithm; and
S3, performing principal component analysis (PCA), performing matrix multiplication of the standardized dataset and feature vectors to obtain PC scores, and taking a sum of scores of first three PCs, PC1, PC2, and PC3, as a metabolic subtype-related prognostic gene score
$$( MSPG - score ) : MSPG = \sum ( PC 1 + PC 2 + PC 3 ) .$$

6. The construction method according to claim 5, wherein in step S2, the genes comprise tetratricopeptide repeat domain 28 (TTC28), glycoprotein A33 (GPA33), phosphodiesterase 7B (PDE7B), sodium voltage-gated channel beta subunit 4 (SCN4B), lamin (LMNB2), hepatocyte nuclear factor 4 gamma (HNF4G), galectin (LGALS3), secreted protein acidic and rich in cysteine-like 1 (SPARCL1), cytidine diphosphate-diacylglycerol synthase 1 (CDS1), zinc finger protein 532 (ZNF532), and furry (FRY).

7. A construction method for a GC prognostic model based on a molecular subtyping method for GC according to claim 2, comprising the following steps:
S1, acquiring overlapped differentially expressed genes (DEGs) among the three MSC subtypes on the basis of a subtyping method, and performing prognostic analysis of each gene using a univariate Cox regression model;
S2, extracting genes with significant prognostic significance using a random forest algorithm; and
S3, performing principal component analysis (PCA), performing matrix multiplication of the standardized dataset and feature vectors to obtain PC scores, and taking a sum of scores of first three PCs, PC1, PC2, and PC3, as a metabolic subtype-related prognostic gene score
$$( MSPG - score ) : MSPG = \sum ( PC 1 + PC 2 + PC 3 ) .$$

8. The construction method according to claim 7, wherein in step S2, the genes comprise tetratricopeptide repeat domain 28 (TTC28), glycoprotein A33 (GPA33), phosphodiesterase 7B (PDE7B), sodium voltage-gated channel beta subunit 4 (SCN4B), lamin (LMNB2), hepatocyte nuclear factor 4 gamma (HNF4G), galectin (LGALS3), secreted protein acidic and rich in cysteine-like 1 (SPARCL1), cytidine diphosphate-diacylglycerol synthase 1 (CDS1), zinc finger protein 532 (ZNF532), and furry (FRY).

9. A construction method for a GC prognostic model based on a molecular subtyping method for GC according to claim 3, comprising the following steps:
S1, acquiring overlapped differentially expressed genes (DEGs) among the three MSC subtypes on the basis of a subtyping method, and performing prognostic analysis of each gene using a univariate Cox regression model;
S2, extracting genes with significant prognostic significance using a random forest algorithm; and
S3, performing principal component analysis (PCA), performing matrix multiplication of the standardized dataset and feature vectors to obtain PC scores, and taking a sum of scores of first three PCs, PC1, PC2, and PC3, as a metabolic subtype-related prognostic gene score
$$( MSPG - score ) : MSPG = \sum ( PC 1 + PC 2 + PC 3 ) .$$

10. The construction method according to claim 9, wherein in step S2, the genes comprise tetratricopeptide repeat domain 28 (TTC28), glycoprotein A33 (GPA33), phosphodiesterase 7B (PDE7B), sodium voltage-gated channel beta subunit 4 (SCN4B), lamin (LMNB2), hepatocyte nuclear factor 4 gamma (HNF4G), galectin (LGALS3), secreted protein acidic and rich in cysteine-like 1 (SPARCL1), cytidine diphosphate-diacylglycerol synthase 1 (CDS1), zinc finger protein 532 (ZNF532), and furry (FRY).

11. A construction method for a GC prognostic model based on a molecular subtyping method for GC according to claim 4, comprising the following steps:
S1, acquiring overlapped differentially expressed genes (DEGs) among the three MSC subtypes on the basis of a subtyping method, and performing prognostic analysis of each gene using a univariate Cox regression model;
S2, extracting genes with significant prognostic significance using a random forest algorithm; and
S3, performing principal component analysis (PCA), performing matrix multiplication of the standardized dataset and feature vectors to obtain PC scores, and taking a sum of scores of first three PCs, PC1, PC2, and PC3, as a metabolic subtype-related prognostic gene score
$$( MSPG - score ) : MSPG = \sum ( PC 1 + PC 2 + PC 3 ) .$$

12. The construction method according to claim 11, wherein in step S2, the genes comprise tetratricopeptide repeat domain 28 (TTC28), glycoprotein A33 (GPA33), phosphodiesterase 7B (PDE7B), sodium voltage-gated channel beta subunit 4 (SCN4B), lamin (LMNB2), hepatocyte nuclear factor 4 gamma (HNF4G), galectin (LGALS3), secreted protein acidic and rich in cysteine-like 1 (SPARCL1), cytidine diphosphate-diacylglycerol synthase 1 (CDS1), zinc finger protein 532 (ZNF532), and furry (FRY).

13. One or more of applications of a molecular subtyping method for GC according to claim 1, comprising:
(a) studies on biological mechanisms related to GC;
(b) prognostic assessment of patients with GC or preparation of products for the prognostic assessment of patients with GC;
(c) related genomic variation studies;
(d) proteomic and phosphoproteomic studies;
(e) prediction of immune response and therapeutic benefit from the therapy using an immune checkpoint inhibitor (ICI) or preparation of products predicting immune response and therapeutic benefit from the therapy using ICI;
(f) studies on copper-related proteins of GC cells; and
(g) studies on drug sensitivity of GC cells.

14. The applications according to claim 13, wherein
in the (b), the prognostic assessment of patients with GC comprises at least survival assessment of the patients with GC;
in the (e), the ICI is specifically a PD-1/PD-L1 inhibitor; and
in the (f), the copper-related proteins of GC cells comprise, but are not limited to, ferredoxin 1 (FDX1), lipoic acid synthetase (LIAS), dihydrolipoamide dehydrogenase (DLD), dihydrolipoamide S-succinyltransferase (DLST), dihydrolipoamide S-acetyltransferase (DLAT), pyruvate dehydrogenase E1 subunit alpha 1 (PDHA1), pyruvate dehydrogenase E1 subunit beta (PDHB), and glutaminase (GLS) proteins.

15. One or more of applications of a molecular subtyping method for GC according to claim 2, comprising:
(a) studies on biological mechanisms related to GC;
(b) prognostic assessment of patients with GC or preparation of products for the prognostic assessment of patients with GC;
(c) related genomic variation studies;
(d) proteomic and phosphoproteomic studies;
(e) prediction of immune response and therapeutic benefit from the therapy using an immune checkpoint inhibitor (ICI) or preparation of products predicting immune response and therapeutic benefit from the therapy using ICI;
(f) studies on copper-related proteins of GC cells; and
(g) studies on drug sensitivity of GC cells.

16. The applications according to claim 15, wherein
in the (b), the prognostic assessment of patients with GC comprises at least survival assessment of the patients with GC;
in the (e), the ICI is specifically a PD-1/PD-L1 inhibitor; and
in the (f), the copper-related proteins of GC cells comprise, but are not limited to, ferredoxin 1 (FDX1), lipoic acid synthetase (LIAS), dihydrolipoamide dehydrogenase (DLD), dihydrolipoamide S-succinyltransferase (DLST), dihydrolipoamide S-acetyltransferase (DLAT), pyruvate dehydrogenase E1 subunit alpha 1 (PDHA1), pyruvate dehydrogenase E1 subunit beta (PDHB), and glutaminase (GLS) proteins.

17. One or more of applications of a molecular subtyping method for GC according to claim 3, comprising:
(a) studies on biological mechanisms related to GC;
(b) prognostic assessment of patients with GC or preparation of products for the prognostic assessment of patients with GC;
(c) related genomic variation studies;
(d) proteomic and phosphoproteomic studies;
(e) prediction of immune response and therapeutic benefit from the therapy using an immune checkpoint inhibitor (ICI) or preparation of products predicting immune response and therapeutic benefit from the therapy using ICI;
(f) studies on copper-related proteins of GC cells; and
(g) studies on drug sensitivity of GC cells.

18. The applications according to claim 17, wherein
in the (b), the prognostic assessment of patients with GC comprises at least survival assessment of the patients with GC;
in the (e), the ICI is specifically a PD-1/PD-L1 inhibitor; and
in the (f), the copper-related proteins of GC cells comprise, but are not limited to, ferredoxin 1 (FDX1), lipoic acid synthetase (LIAS), dihydrolipoamide dehydrogenase (DLD), dihydrolipoamide S-succinyltransferase (DLST), dihydrolipoamide S-acetyltransferase (DLAT), pyruvate dehydrogenase E1 subunit alpha 1 (PDHA1), pyruvate dehydrogenase E1 subunit beta (PDHB), and glutaminase (GLS) proteins.

19. One or more of applications of a molecular subtyping method for GC according to claim 4, comprising:
(a) studies on biological mechanisms related to GC;
(b) prognostic assessment of patients with GC or preparation of products for the prognostic assessment of patients with GC;
(c) related genomic variation studies;
(d) proteomic and phosphoproteomic studies;
(e) prediction of immune response and therapeutic benefit from the therapy using an immune checkpoint inhibitor (ICI) or preparation of products predicting immune response and therapeutic benefit from the therapy using ICI;
(f) studies on copper-related proteins of GC cells; and
(g) studies on drug sensitivity of GC cells.

20. The applications according to claim 19, wherein
in the (b), the prognostic assessment of patients with GC comprises at least survival assessment of the patients with GC;
in the (e), the ICI is specifically a PD-1/PD-L1 inhibitor; and
in the (f), the copper-related proteins of GC cells comprise, but are not limited to, ferredoxin 1 (FDX1), lipoic acid synthetase (LIAS), dihydrolipoamide dehydrogenase (DLD), dihydrolipoamide S-succinyltransferase (DLST), dihydrolipoamide S-acetyltransferase (DLAT), pyruvate dehydrogenase E1 subunit alpha 1 (PDHA1), pyruvate dehydrogenase E1 subunit beta (PDHB), and glutaminase (GLS) proteins.
